(19) United States
(12) Patent Application Publication
(10) Pub. No.: US 2020/0347347 A1
(43) Pub. Date: Nov. 5, 2020

(54) COMPOSITIONS AND METHODS FOR IMPROVED GAMETE VIABILITY AND FUNCTION

(71) Applicant: Premium Genetics [UK] Ltd., London, Cheshire (GB)

(72) Inventor: Elon C. ROTI ROTI, DeForest, WI (US)

(21) Appl. No.: 16/760,398

(22) PCT Filed: Oct. 30, 2018
(86) PCT No.: PCT/IB2018/001403

§ 371 Date: Apr. 29, 2020

Related U.S. Application Data
(60) Provisional application No. 62/578,959, filed on Oct. 30, 2017.

(51) Int. Cl. C12N 5/00; C12N 5/076; A01N 1/02

(52) U.S. Cl. CPC C12N 5/0018; C12N 5/061; C12N 2500/16; C12N 2500/14; A01N 1/0284

(57) ABSTRACT

The present specification provides for a media composition that provides enhanced viability and activity to sperm cells.

### CROSS-REFERENCE TO RELATED APPLICATIONS

The application claims priority from U.S. Provisional Application No. 62/578,959 filed Oct. 30, 2017, the content of which is herein incorporated in its entirety.

### FIELD OF THE INVENTION

The present disclosure relates to the field of animal husbandry and breeding. In particular, the present disclosure includes improved formulations of media for use with ejaculate and sperm cell samples, and in fertilization processes. Such media formulations provide improved viability of sperm cells, enhanced sperm cell function, including motility and fertility, and enhanced zygote formation (i.e., fertilization).

### BACKGROUND OF THE INVENTION

An important aspect of animal husbandry, particularly in agriculture, is the collection and use of sperm cells (spermatozoa). Generally, sperm cells are collected in the form of raw ejaculate from male animals. Subsequent use and manipulation of the sperm cells requires that the viability and function of the cells be maintained for hours or even days.

A substantial problem with the manipulation of reproductive cells in vitro can be a significant loss of reproductive cell characteristics such as alteration of the lipid bilayer, alteration of cellular organelles, cell apoptosis, or cell necrosis, and decreased motility of sperm cells. The loss of reproductive cell characteristics can result in decreased fertility or decreased viability of the reproductive cells, or both. A decrease in the viability or fertility of reproductive cells can be a significant disadvantage in the context of the preparation, cooling, freezing, cooled or frozen storage, thawing or thawed storage of sperm cells contained in artificial insemination straws (or other containers or vessels), the artificial insemination of animals, the preparation, manipulation, cooling, freezing, cooled or frozen storage, the thawing or thawed storage of oocytes, the in vitro fertilization of oocytes, or the like.

This problem can be further exacerbated in the context of recent advances in the flow analysis or the flow sort of sperm cells to obtain sex-selected populations of sperm cells (populations of sperm cells bearing predominantly an X-chromosome or a Y-chromosome). Because flow analyzed or flow-sorted sperm cells undergo an increased number of manipulations including staining the nuclear DNA, flow analysis, flow sorting, and collecting the desired number of sperm cells, the resulting flow-sorted sperm cells collected have an increased likelihood of a significant loss of in vivo reproductive cell characteristics.

The sexing process subjects the sperm to cellular insults (Alvarz and Storey, 1992). These stresses decrease the viable cell population, and rapid losses are expected during at least two steps: incubation (at about 19° C.) before staining and sexing; and during freezing for long-term storage. Induced oxidative DNA damage in sperm decreases fertilization rates and high levels of damage cause developmental arrest after embryonic transcript activation (Aitken et al., 2009; Fatehi et al., 2006).

Assisted reproductive technology (ART) includes such techniques as in vitro fertilization (IVF), artificial insemination (AI), intracytoplasmic sperm injection (ICSI) (and other techniques using enucleated cells) and multiple ovulation and embryo transfer (MOET) (as well as other embryo transfer techniques), is used across the animal kingdom, including humans and other animals. ART methods are usually expensive, time-consuming and variably successful given the inherent fragility of gametes and embryos when outside of their natural environments. Furthermore, the use of ART within the animal breeding industry in a commercially feasible manner is additionally challenging due to the limited availability of genetically desirable gametes and zygotes. One way to lower the cost of ART and to improve its commercial feasibility is to increase the efficiency of the involved processes by improving the viability and overall quality of gametes and zygotes. Although there is has been a growing interest in this field over the course of the last decade or so, there still remains a strong need to increase the overall quality of gametes and zygotes for use in ART, especially when breeding focuses on pre-natal gender selection, including improving their viability (in the case of gametes and zygotes), their motility and fertility (in the case of sperm cells), as well as other longevity characteristics.

For example, in IVF, the percentage of zygotes that develop into embryos using existing techniques is relatively low; this high rate of loss significantly increases the cost of embryos and related services to end-users and decreases the effective availability of high-quality embryos. These cost and availability issues can be further exacerbated by subsequent post-embryo handling through cryopreservation as well as non-frozen transport. Cryopreservation of embryos is limited by the success rate of embryo production as well as blastocyst growth in vitro. Currently, only a marginal percentage of IVF embryos are suitable for cryopreservation which adds to the ongoing high cost of ART procedures.

Especially when processing gametes such as flushed oocytes or sperm cells, both conventional and sex-sorted, before their use in ART adds a tremendous amount of stress on the gamete cell and negatively impacts their cellular integrity and membrane structure which in turn is reflected in decreased viability, motility, and fertility. An example of processing gametes prior to their use in ART is the sorting of sperm cells based on sex (known as “gender enrichment” or “sex-sorting”), which is a highly desired procedure to minimize wasted births of the wrong sex for selective breeding in the livestock industry but is often cost prohibitive and can be risky to those with smaller breeding herds.

The popular flow cytometry-based sex-sorting process severely stresses and damages the cells and produces a low percentage of useful sperm, which although capable of fertilizing matured oocytes, have reduced viability, motility and fertility after the sex-sorting process. Typically, sex-sorting involves many harsh steps including but not limited to the initial collection and handling of sperm ejaculate which naturally starts to deteriorate rapidly upon collection; the staining of sperm cells which involves binding of an excitable dye to the DNA or a harmful membrane selection procedure, the physical sorting of the sperm cells using high energy fluorescence that physically energizes the dye that is bound to the DNA, forced orientation through a narrow orifice, and application of an electrical charge to the cell, the physical collection of the cells into a container which often shocks the fragile cell upon contact, the osmotic stresses associated with dilution of the sperm droplet in collection media, and the storage of the sorted sperm usually by freezing which is well known to raise havoc with the cell's membrane systems. Each step places the processed sperm under abnormal stress which diminishes the overall motility, viability and/or fertility of the sperm. The result can lead to less efficient samples for use in ART, such as IVF and AI, and other types of subsequent or further processing.

Even non-sorted processed sperm exhibits significant losses in fertility, viability, and motility when being collected, handled and transported without freezing, and noticeably experiences significant stress when mixed with cryoprotectant and is frozen and thawed. Many in the field have tried to improve methods for the use on unsorted, conventional semen to minimize the loss in the handling processes associated with in vitro handling, preservation and use of semen samples.

Regardless of the processing, sperm lose their potential to fertilize when exposed to: elevated temperatures, abnormal buffers, stains, altered pH systems, physical pressurized orientation as when forced through a nozzle or when oscillated to form drops in a flow cytometer, radiation used to illuminate the DNA binding dye, physical stressors associated with separation and collection techniques, cryoprotectants, freezing, thawing and micromanipulation by the handler.

Other commercially available media do not provide the necessary performance characteristics when used with sperm cells. Commercial media may not allow for adequate maintenance of viability and/or activity of sperm. Additionally, commercially available media also can cause interference with downstream use of the sperm, such as during sex-sorting or in IVF or AI.

Minimizing cell stressors increases the number of sperm surviving the sexing process (or other manipulations), and therefore increases the availability of sexed semen for farmers. One approach to minimize these stressed is to add semen extender to raw ejaculate to preserve viability and motility, which may, in turn, improve cell tolerance to the stresses of the sexing process. An extender that preserves the viable, motile sperm population eligible for sexing can significantly reduce the cost of sexed semen to farmers by increasing yield in a few ways. Currently significant portion (approximately 50%) of viable cells packaged in the insemination straws during cryopreservation, but an extender could increase the number of cells that survive freezing by minimizing upstream stressors.

To date, there remains a significant need for improvement in the compositions and methods involved in the routine handling of fragile gametes during in vitro processing, especially during the harsh processing associated with the sex-sorting of sperm, whereby the result is a reproducible improvement on the viability, motility, and fertility of sperm cells and embryos. There remains a continuing need to improve current methods of ART to reduce the cost and to make the procedures more dependable and commercially feasible to those on a tight budget, especially those smaller breeders who view sex-selection breeding as a high risk and expensive option.

The ideal extender formulation must maintain a high motile sperm population and must not interfere with the ability to separate the X and Y populations using a fluorescent DNA stain, which is required to separate the two cell populations on the sexing cytometry instruments.

### SUMMARY OF THE INVENTION

Certain embodiments of the claimed invention are summarized below. These embodiments are not intended to limit the scope of the claimed invention, but rather serve as brief descriptions of possible forms of the invention. The invention may encompass a variety of forms which differ from these summaries.

One aspect of the disclosure relates to a media formulation comprising a basic salt media. In another aspect the basic salt media comprises at least one component selected from the group consisting of sodium chloride (NaCl), potassium chloride (KCl), calcium chloride dihydrate (CaCl₂.2H₂O), magnesium chloride, hexahydrate (MgCl₂.6H₂O), sodium bicarbonate (NaHCO₃), sodium phosphate monobasic dehydrate (NaH₂PO₄.2H₂O), Potassium dihydrogen phosphate (KH₂PO₄), fructose, sorbitol, bovine serum slbumin (BSA), TRIS base, citric acid, and combinations thereof.

In another aspect, the media formulation comprises an additive. In yet another aspect, the additive is selected from the group consisting of phosphatidylserine (PS), decursin, zinc chloride, coenzyme Q10, coumarin compounds, pyranocoumarin compounds, NSAID, linolenic acid, fatty acids, D-aspartic acid, sodium fluoride, and combinations thereof. In yet another aspect, the NSAID is acetylsalicylic acid. In yet another aspect, the pyranocoumarin compound is decursin. In yet another aspect, the media formulation comprises a basic salt media and an additive.

In another aspect of the disclosure, the media formulation enhances activity of mammalian reproductive cells, enhances zygote/blastocyst formation from germ cells (i.e., increased fertilization), enhances the viability, mobility, and/or fertility of sperm cells, maintains sperm in a fertilization competent state, or alleviates cell loss or DNA damage due to freeze-thaw process. Fertilization competence is the capability of sperm cells exposed to the media formulation of the present invention for producing pregnancies via artificial insemination, and fertilization, cleavage, or blastocyst conversion both in vitro and in vivo.

In another aspect, the media formulation extends cell viability for at least 24 hours.

In yet a further aspect, the mammalian reproductive cells are selected from the group consisting of gametes, haploid cells, germ cells, sex cells, sperm cells, and egg cells. In yet another aspect, the media formulation is used in a method to enhance the viability or fertility of sperm cells. In yet another aspect, the mammalian reproductive cells can be derived from ejaculate from male mammal.

One aspect of the disclosure relates to a composition comprising the media formulation and ejaculate from a male mammal. In another aspect the media formulation comprises the basic salt media. In another aspect the media formulation comprises an additive. In yet another aspect, the media formulation comprises a basic salt media and an additive. In yet another aspect the composition is cryopreserved.

One aspect of the disclosure relates to a method of processing mammalian reproductive cells comprising the steps of providing a mammalian reproductive cells sample, processing the mammalian reproductive cells sample, and adding the media formulation of the present invention. In another aspect the media formulation comprises the basic salt media. In another aspect the media formulation comprises an additive. In yet another aspect, the media formulation comprises a basic salt media and an additive. In yet another aspect, the processing comprises at least one step selected from the group consisting of collecting a semen sample, sexing, sorting, separating, freezing, artificial insemination, in vitro fertilization, cooling, transport, and related processes. In yet a further aspect, the sexing is accomplished via droplet sorting, mechanical sorting, micro fluidic processing, microchip processing, jet and air processing, flow cytometry processing, and laser ablation. In yet another aspect, the male mammal is a bull or boar. In yet a further aspect, the processed mammalian reproductive cells are gathered in a container, tube, or straw. In a further aspect, the mammalian reproductive cells are selected from the group consisting of gametes, haploid cells, germ cells, sex cells, sperm cells, and egg cells. In yet another aspect, a sperm cell composition is produced by this processing method.

One aspect of the disclosure relates to a method of processing a sperm sample. In some aspects, this method comprises obtaining an ejaculate from a male mammal and combining said ejaculate with the media formulation. In another aspect the media formulation comprises the basic salt media. In another aspect the media formulation comprises an additive. In yet another aspect, the media formulation comprises a basic salt media and an additive.

One aspect of the disclosure relates to a method of fertilizing one or more eggs comprising the step of providing an egg obtained from a female mammal, providing the sperm cell composition from a male mammal of the same species as the female mammal, and mixing one or more eggs with the sperm composition. In some aspect, the sperm cell composition is produced by the processing methods disclosed. In yet another aspect, the sperm cell composition is mixed with the media formulation. In another aspect the media formulation comprises the basic salt media. In another aspect the media formulation comprises an additive. In yet another aspect, the media formulation comprises a basic salt media and an additive. In yet another aspect, the male mammal is a bull or boar.

One aspect of the disclosure relates to a method of producing an embryo comprising using a sperm cell composition from a male mammal for assisted reproductive techniques. In some aspect, the sperm cell composition is produced by the processing methods disclosed. In yet another aspect, the sperm cell composition is mixed with the media formulation. In another aspect the media formulation comprises the basic salt media. In another aspect the media formulation comprises an additive. In yet another aspect, the media formulation comprises a basic salt media and an additive. In yet another aspect, the male mammal is a bull or boar. In some aspects, the assisted reproductive technique is selected from the group consisting of in vitro fertilization (IVF), artificial insemination (AI), intracytoplasmic sperm injection (ICSI), multiple ovulation and embryo transfer (MOET), and other embryo transfer techniques.

In a further aspect, the method further comprises sexing the sperm sample. One aspect of the disclosure relates to a method of sexing a sperm cell population comprising the steps of providing a sperm cell sample, sexing the sperm cell sample into at least one subpopulation, and adding at least one additive selected from the group consisting of antioxidants, phosphatidylserine (PS), decursin, zinc chloride, coenzyme Q10, acetylsalicylic acid, linolenic acid, fatty acids, D-aspartic acid, sodium fluoride, and combinations thereof to the sperm cell sample. In a further aspect, the sperm cell population further comprises seminal fluid components and/or raw ejaculate. In yet another aspect, the sexed subpopulation comprises at least one gender enriched population of X-chromosome bearing or Y-chromosome bearing sperm cells. In a further aspect, the method comprises combining a sexed sperm sample with the media formulation. In another aspect the media formulation comprises the basic salt media. In another aspect the media formulation comprises an additive. In yet another aspect, the media formulation comprises a basic salt media and an additive. In yet another aspect, a sperm cell composition is produced by this method.

In a further aspect, collecting subpopulations of the sperm cell populations includes gathering both the selected and the unselected subpopulations together, usually in a container, a tube or a straw. It could be further defined as the stream of population of sperm cells are not physically subdivided prior to and subsequent to ablation of a subpopulation of sperm cells and that both subpopulations exit together and are gathered.

One aspect of this disclosure relates to a method of fertilizing one or more eggs comprising the steps of providing an egg obtained from a female mammal, providing the sexed sperm cell composition of claim from a male mammal of the same species as the female mammal, and mixing one or more eggs with the sperm composition.

Yet another aspect is a method of producing an embryo comprising using a sexed sperm cell composition from a male mammal for assisted reproductive techniques. In some aspects, the sexed sperm cell composition is produced by the processing methods disclosed. In yet another aspect, the sperm cell composition is mixed with the media formulation. In another aspect the media formulation comprises the basic salt media. In another aspect the media formulation comprises an additive. In yet another aspect, the media formulation comprises a basic salt media and an additive. In yet another aspect, the male mammal is a bull or boar. In some aspects, the assisted reproductive technique is selected from the group consisting of in vitro fertilization (IVF), artificial insemination (AI), intracytoplasmic sperm injection (ICSI), multiple ovulation and embryo transfer (MOET), and other embryo transfer techniques.

One aspect of the disclosure relates to a method of fertilization, comprising providing an egg obtained from a female mammal; providing a sperm sample obtained from a male mammal of the same species as said female mammal, said sperm sample comprising sperm cells and at least one additive selected from the group consisting of phosphatidylserine (PS), one or more coumarin compounds or pyranocoumarin compounds, zinc chloride, coenzyme Q10, one or more NSAID, linolenic acid, fatty acids, D-aspartic acid, sodium fluoride, and combinations thereof; and fertilizing said egg with said sperm sample. In a further aspect, the fertilization comprises in vitro fertilization. In another aspect, the fertilization comprises artificial insemination (AI). In another aspect, the sperm cell composition further comprising seminal fluid components and/or raw ejaculate. In yet a further aspect, the sperm sample may be sex-selected, and comprise an increased proportion of either X-chromosome bearing or Y-chromosome bearing sperm cells. One aspect of the disclosure relates to a media formulation for enhancing viability of mammalian reproductive cells wherein said media comprises phosphatidylserine (PS). In further aspects, the media formulation further comprises sodium fluoride. In yet another aspect, the media formulation further comprising decursin, zinc chloride, coenzyme Q10, acetylsalicylic acid, linolenic acid, fatty acids, D-aspartic acid, or combinations thereof.

One aspect of the disclosure relates to a method of preserving a sperm sample, comprising obtaining an ejaculate from a male mammal and combining said ejaculate with a media formulation comprising phosphatidylserine (PS). In another aspect, the media formulation further comprises sodium fluoride. In yet another aspect, the media formulation further comprises decursin, zinc chloride, coenzyme Q10, acetylsalicylic acid, linolenic acid, fatty acids, D-aspartic acid, or combinations thereof. In another aspect, the basic salt media is formulated similar to CEP2 (cauda epididymal plasma) media (Verberckmoes, 2004). In another aspect this basic salt media is supplemented with additional formulation, herein referred to as spermatozoa viability and fertility enhancing media (SVFEM). Cells extended in SVFEM for ≥24 hours exhibited a motile population within ten percentage points of paired non-extended ejaculate samples assessed immediately after collection. Cells extended in SVFEM produced a sexed, cryopreserved semen product that meets outgoing quality control standards. Suggesting improved tolerance to cryopreservation, the number of motile cells per straw post-thaw (the insemination dose) increased in SVFEM-extended split ejaculates compared to non-extended controls sexed the same day and packaged at the same cell concentration pre-freeze.

Preliminary analysis of the sperm response to SVFEM extender detailed above confirmed that sperm are live and motile after a 24-hour incubation.

In some aspects, the components of the media formulation are grouped into a concentrated solution and stored. These stock solutions can be stored as a liquid, frozen, or lyophilized. In some aspects, the media formulation is prepared from a stock solution. In a further aspect, the media formulation is prepared from made from a multi-component stock solution, including, but not limited to a two-component stock solution and alternatively a three-component stock solution.

Other aspects of the disclosure comprise a kit for supplementing media. In some aspects, the kit comprises at least one additive selected from the group consisting of antioxidants, phosphatidylserine (PS), decursin, zinc chloride, coenzyme Q10, coumarin compounds, pyranocoumarin compounds, NSAIDs, linolenic acid, fatty acids, D-aspartic acid, sodium fluoride, and combinations thereof. Kits may further comprise a tube or container containing one or more components, additives, salt bases, or media formulations. Kits may further comprise additional tubes or containers containing additional components, additives, salt bases, or media formulations. Kits may also comprise instructions for use, such as instructions for preparing a medium, processing mammalian reproductive cells, fertilizing one or more eggs, or producing embryos.

Other aspects will be apparent to one of skill in the art upon review of the description and exemplary aspects and embodiments that follow.

### BRIEF DESCRIPTION OF THE DRAWINGS

To illustrate the disclosure, depicted in the drawings are certain features of the aspects and embodiments of the disclosure. However, the disclosure is not limited to the precise arrangements and instrumentalities of the aspects depicted in the drawings.

FIG. 1 is a plot that quantifies the percentage of progressively motile cells present in semen stored at 19° C. for 0 or 24 hours and demonstrates SVFEM extension preserves the motile cell population within ten percentage points of measured incoming values. Progressive motile % was determined using a CASA system. N=10 paired ejaculates.

FIG. 2 shows that sodium fluoride supplemented media, when compared to the base SVFEM media formulation without sodium fluoride (NaF) showed a minor decrease in % progressive motile cells after staining at time 24. The graph shows the data from SVFEM media supplemented with 3 mM NaF normalized to SVFEM with 0 mM NaF. At Time 0, most values remain the same as the control SVFEM group, but that by 24 hours they have all fallen at least 20% below the values in the paired SVFEM group. N=8 unique sire's ejaculates. Values are normalized to the 0 mM NaF control group. This decrease indicated that NaF supplementation in the SVFEM formula was not the optimal formulation.

FIG. 3 exhibits quantification of a number of the concentration of progressively motile cells per insemination dose after freeze-thaw in SVFEM treated ejaculates sexed at six different timepoints over 2 days. The gray line represents the failure threshold for progressive cells, 1M/mL. SVFEM treated ejaculates survive the freeze-thaw process after incubation for up to 32 hours before the sexing process begins. Progressive cells per straw are plotted as mean+/−SE. N=24 unique ejaculates from 15 different Holstein (11) and Jersey (4) sires.

FIG. 4 shows the mean blastocyst conversion for three paired ejaculates suggests incubation with SVFEM extender does not affect blastocyst conversion when compared to paired sexed control samples. No statistically significant differences. N=3 paired ejaculates from 3 unique sires.

FIG. 5 shows the diagrammatic outline of the IVF procedure.

FIG. 6 shows representative images for each assessed fertilization efficiency category: A—monospermic as represented by two decondensed pronuclei and two condensed polar bodies, B—polyspermic as categorized by 3 or more decondensed pronuclei, C—unfertilized with no present pronuclei, D—other, obscuring fluorescence from cumulus cells prevent adequate scoring. All zygotes imaged are from one treatment group, control sexed semen.

FIG. 7 shows percent polyspermic fertilizations calculated out of the total number of presumptive zygotes scored shows a significant increase in T₂₄ SVFEM. Values represent the average of all assessed zygotes from that group. There are no significant differences between the groups as measured by a one-way ANOVA on natural log transformed data overall p-value is 0.279. P-values for T₀ compared to controls is 0.450 and T₂₄ compared to controls is 0.483. N=50 unique ejaculates.

FIG. 8 shows percent monospermic fertilizations calculated out of the total number of presumptive zygotes scored shows no significant differences. Values are not normalized and represent the average of all assessed zygotes from that group. Overall one-way ANOVA on arcsine transformed data gives p=0.197. N=50 unique ejaculates.

FIG. 9 shows percent unfertilized oocytes calculated out of the total number of presumptive zygotes scored show a significant decrease in both SVFEM treated groups. Values are not normalized and represent the average of all assessed zygotes from that group. Overall there are significant differences as measured by a one-way ANOVA p=0.002. Bonferroni test specifies the specific difference between the control and SVFEM T₀ p=0.004 and between control and T₂₄ p=0.013. N=50 unique ejaculates

FIG. 10 shows cleavage percent shows significant increases in the percent of cleaved zygotes in both SVFEM treated groups. Significant differences are seen in a one-way ANOVA of arcsine transformed data p=0.0004. Bonferroni test identifies two significant differences in the groups; control is different from T₀ p=0.002, and control from T₂₄ p=0.001. N=60 unique ejaculates.

FIG. 11 shows the average percent of blastocysts day 7 shows significant increases in the percent of blastocysts per oocyte in both SVFEM groups. Overall significant differences are seen in one-way ANOVA of arcsine transformed data p=0.0008. Bonferroni analysis identified differences between the treatments and the control. T₀ compared to control gives p=0.008 and T₂₄ compared to control gives p=0.002. N=60 unique ejaculates.

FIG. 12 shows the average percent of blastocysts day 8 show an increase in the percent of blastocysts per oocyte in T₀ SVFEM group. Overall significant differences are seen in one-way ANOVA p=0.031. Bonferroni analysis identified differences between T₀ SVFEM and control giving a p-value of 0.037, and no differences between T₂₄ and control, p=0.143. N=60 unique ejaculates.

FIG. 13 shows the difference in average blastocyst development between day 7 and day 8 shows no significant differences between the groups, overall ANOVA p-value 0.723. N=60 unique ejaculates.

FIG. 14 shows an average number of blastocysts day 8 showing the outcomes across all three treatment groups for each breed; no consistent trends are evident in these graphs. A is control, L is T₀ SVFEM, S is T₂₄ SVFEM. No consistent trends marked in analysis with ANOVA. N=20 unique bulls.

FIG. 15 shows average blastocysts day 8 separated by star ranking of bull; no consistent trends evident on the graphs. A is control, L is T₀ SVFEM, S is T₂₄ SVFEM. No consistent trends calculated in analysis with ANOVA. N=20 unique bulls.

FIG. 16 shows average blastocysts day 8 divided by age group; no consistent trends evident in graphs. A is control, L is T₀ SVFEM, S is T₂₄ SVFEM. No consistent trends calculated in ANOVA analysis. N=20 unique bulls.

FIG. 17 shows average cleaved percent normalized to the conventional un-sexed control semen shows both SVFEM groups and the non-extended control cleave significantly less than the conventional control. Mean values on the graph represent the percent difference from the average value in the conventional semen sample. Both treatment groups and the sexed semen control are significantly different in a one-way ANOVA p<0.001. N=29 unique ejaculates.

FIG. 18 shows average blastocyst day 7 percent normalized to the conventional un-sexed control semen show both SVFEM groups and the non-extended semen convert to blastocysts on day 7 significantly less than conventional control semen. Mean values on the graph represent the percent difference from the average value in the conventional semen sample. Both treatment groups and the sexed semen control are significantly different in a one-way ANOVA p<0.001. N=29 unique ejaculates.

FIG. 19 shows average blastocyst day 8 percent normalized to the conventional un-sexed control semen show that both SVFEM groups and the non-extended control convert to blastocysts on day 8 significantly less than the conventional control semen. Mean values on the graph represent the percent difference from the average value in the conventional semen sample. Both treatment groups and the sexed semen control are significantly different in a one-way ANOVA p<0.001. N=29 unique ejaculates.

FIG. 20 exhibits images showing the lack of DNA decondensation and migration from representative comet assay images. A and C are 200 μM H₂O₂ treated cells, and B and D are control diH₂O treated cells. C and D were mechanically homogenized before gel embedding, and A and B were not. In A and B bright field shows sperm head shapes with DNA tightly compact within, C and D do not have structures visible in bright field images, but DNA is still tightly compact in an ovoid shape. Fluorescence was dim in C and D and was washed out in bright field converged images.

FIG. 21 shows a comparison in motility after 24 hr of storage between SVFEM and a control.

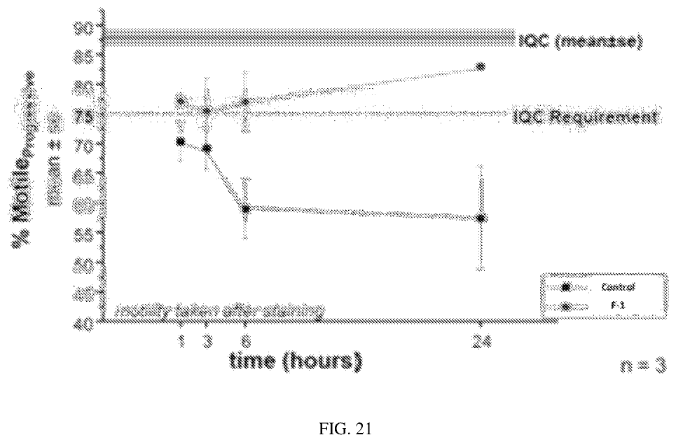

FIG. 22 shows the effect of NaF concentration in SVFEM (F-1) on a number of motile cells.

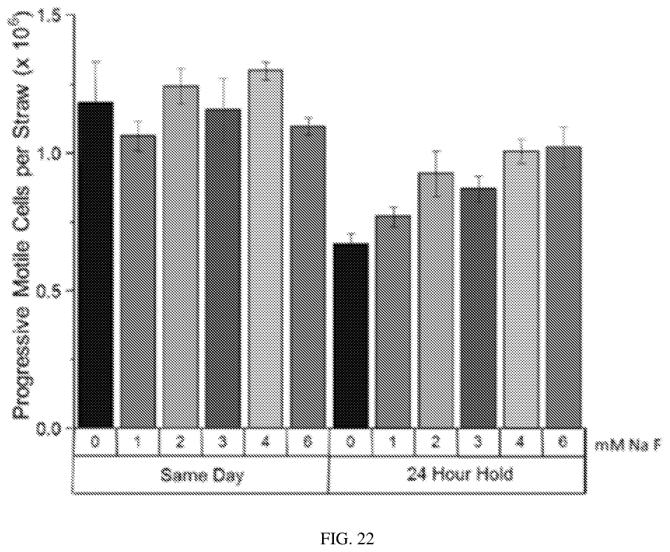

FIG. 23 shows an improved yield of motile cells using SVFEM (F-1) for conventionally processed samples.

FIG. 24 shows the effect of 24-hour storage of sperm samples in SVFEM (F-1).

FIG. 25 shows the effect of 24-hour storage of sperm samples using SVFEM (F-1) versus samples using a control media. Samples pre-processing and post-staining are compared.

FIG. 26 shows the effect of 24-hour storage of sperm samples using SVFEM (F-1) versus samples using a control media. Samples are straws of post-processed sperm.

FIG. 27 shows IVF data utilizing samples containing SVFEM (F-1). Sexed samples using a control media are compared to SVFEM (F-1) and also to conventionally processed samples (not sexed).

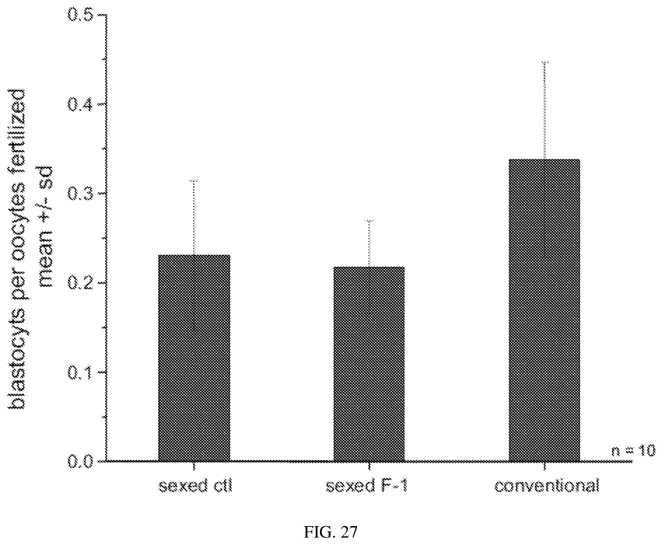

FIG. 28 shows more IVF data utilizing samples containing SVFEM (F-1). Sexed samples using a control media are compared to SVFEM (F-1).

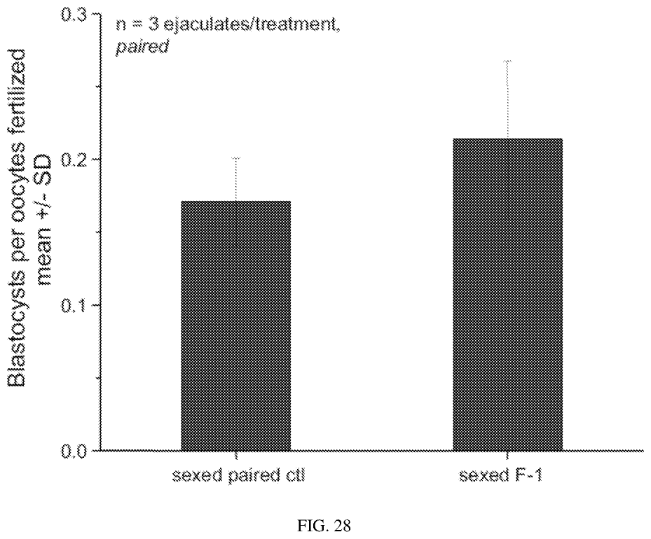

FIG. 29 shows the effect of eliminating components of SVFEM (F-1) on the motility of cells after 24 hours.

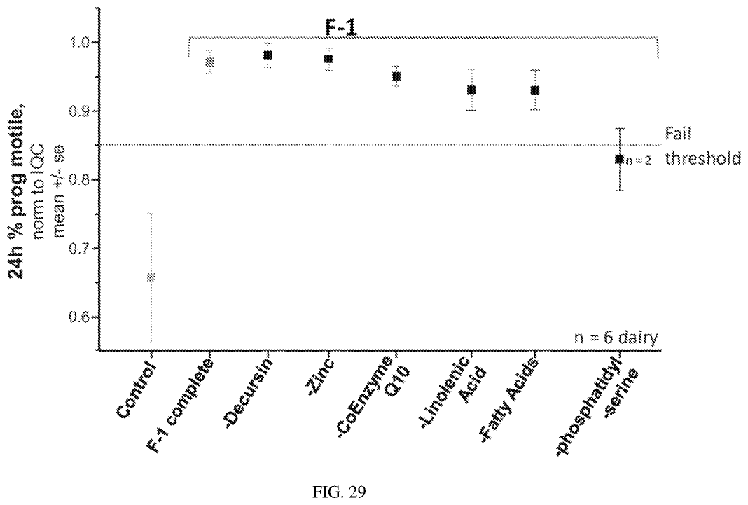

FIG. 30 shows the effectiveness of SVFEM (F-1) made from a three-component stock solution.

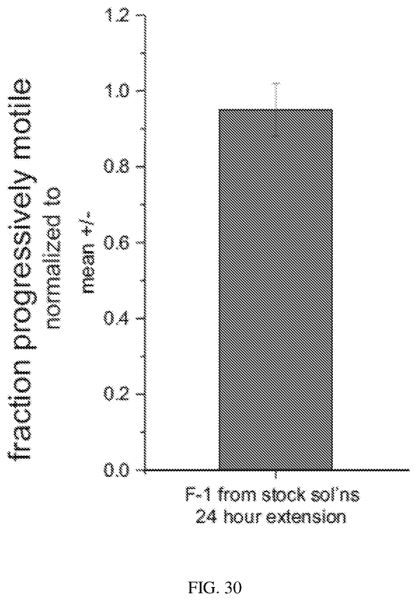

### DETAILED DESCRIPTION

Before continuing to describe various aspects and embodiments in further detail, it is to be understood that this disclosure is not limited to specific compositions or process steps and may vary. As used in this specification and the appended claims, the singular form “a,” “an,” and “the” include plural referents unless the context dictates otherwise. Ranges expressed herein are inclusive.

Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention.

The present disclosure relates to compositions and methods that improve reproductive cell viability and activity. Specifically, in one form the present specification provides and includes media formulations that impart increased activity and viability to reproductive cells, in particular, sperm cells. The present specification in another form provides and includes methods for processing reproductive cell samples, wherein the methods and processes produce samples in which the sperm cells have increased viability and activity. The present specification, in another form, also provides and includes the reproductive cell samples produced by these methods, wherein reproductive cells in the samples have increased viability and activity. In another form, the present specification provides and includes methods using these reproductive cell samples with increased viability and activity, including sexing (selecting X-chromosome bearing or Y-chromosome bearing cells), sorting, separating, freezing, artificial insemination, in vitro fertilization, cooling and transport, and related processes.

The term “sexing” as used herein refers to any process that selects X-chromosome bearing or Y-chromosome bearing sperm cells from a population that comprises a mixture of both X-chromosome and Y-chromosome bearing sperm cells. The sperm cell population can be raw ejaculate, or any other mixture or sperm cells. The sexing process can be accomplished using a number of different techniques, including droplet sorting (described in U.S. Pat. No. [ ]), mechanical sorting, and laser ablation.

The term “reproductive cell” as referred herein is defined as sperm, eggs, and the formation of embryo/blastocyst, also gametes; haploid cells; germ cells; sex cells; sperm cells and egg cells.

The term “medium” or “media” as used herein refers to an essentially liquid composition that may contain nutrients, salts, and other substances or constituents.

The term “ejaculate” as used herein refers to the combination of semen and spermatozoa produced by a male mammal, as released by ejaculation.

The term “seminal fluid components” as referred herein is the substances that make up and/or are commonly found in mammalian semen. Seminal fluid components include, but are not limited to, amino acids, prostate specific antigen, proteolytic enzymes, citric acid, citrate, sialic acid, vitamin C, acid phosphatase, fibrinolysin, lipids, fructose, prostaglandins, phosphorylcholine, glycerophosphocholine, flavins, basic amines such as putrescine, spermine, spermidine and cadaverine, zinc, galactose, mucus and other organic and inorganic constituents.

Sexing procedures disclosedcan be implemented for use with fresh, un-extended ejaculate. However, there are inherent issues to using fresh ejaculate that is not supplemented with an extender, as an ejaculates decline in quality continually after collection. Sperm that undergoes sexing is exposed to numerous insults including temperature swings, high dilution, and pH changes during cell processing. Insults during sexing include shear stress, high fluid pressure, and the high force caused by the sexing process on cytometers (Garner and Seidel, 2003; Garner, 2006). These insults lead to a decrease in the number of cells recovered after processing.

That loss of cells during processing, while detrimental, is not the main source of projected product loss. The major loss is due to fresh ejaculates having a steady increase in dead cell population over time after collection. A major factor of this is that the fresh ejaculates must be stored at close to room temperature, as the sexing process happens at room temperature. Keeping the ejaculate at room temperature, rather than at a cooler temperature that better preserves cell survivability prevents time spent on equilibrations to the cooler temperature and allows sexing of the ejaculates continuously. This also increases the rapidity in which the viable cells are lost before the sexing process begins. This continual loss of viability when paired with the time required to perform the sexing and the packaging steps led to the standard policy of freezing a sexed ejaculate no later than 12 hours after collection.

This policy of having to package an ejaculate no later than 12 hours after collection leaves a lot of ejaculate volume behind due to surplus ejaculate volume. These excess volumes cannot be utilized because fresher ejaculates are being collected before they can be used entirely. Ejaculates are collected every 6 hours, 24 hours a day, to prevent a lapse in instrument running time due to lack of sample. This means that ejaculates from the early morning collection frequently still have viable volume left to run when the second daily collection arrives but are still removed and replaced in favor of the fresher ejaculate because the older sample is not likely to run an additional 6 hours on the instrument. Favoring fresh ejaculates leads to a second major source of loss: downtime on sexing instruments. All ejaculates from a given time point are removed and replaced at the same times each day, meaning instruments are shut down, cleaned, and restarted four times daily. This means that a minimum of 40 minutes of run time is lost, four times daily, for each instrument. These numbers calculated in a number of cells is 46.9×10⁸ skewed cells uncollected per instrument per day; which translates to approximately one thousand insemination doses lost per day.

An extender formulated for use in a sperm sexing facility could help to mitigate losses. First, an extender can slow the decline of sperm cells held at room temperature before sexing, allowing a larger number of ejaculates to be collected at a single time point, and decreasing the number of times per day ejaculates are collected. In this model, instruments would only be shut down to change to a new ejaculate as needed on a bull by bull basis, rather than the entire production floor at once. This would decrease the time it takes to change to a new bull, as it increases the available staff per instrument. Maintaining cell viability also means ejaculates could be run until exhaustion, maximizing the number of sexed sperm obtained per ejaculate, and decreasing the total number of times per day a bull change would be performed per instrument. By mitigating these causes of cell loss, the number of insemination doses produced would increase making the superior product more available to farmers globally.

In addition, a large amount of cell loss is observed during the freeze-thaw process. Both conventional (i.e. non-sexed) and sexed semen are typically frozen in straws for storage and distribution. The straws must be subsequently thawed prior to use for insemination or fertilization. This process of freezing and thawing results in the death of a large proportion of the cells in the straw. The media formulations and processes disclosed herein can be used to alleviate this cell loss due to the freeze-thaw process.

### Enhanced Extension Media Formulation

The enhanced extension media formulation of the present invention provides a number of important benefits: it extends cell viability for at least 24 hours, with a loss of progressively motile cells no greater than ten percentage points; it does not interfere with Hoechst 33342 (or an alternative) staining and red dye viability counterstaining of the cells which is necessary for proper sexing on the cytometers; and it does not negatively interfere with fertilization capacity or embryonic development.

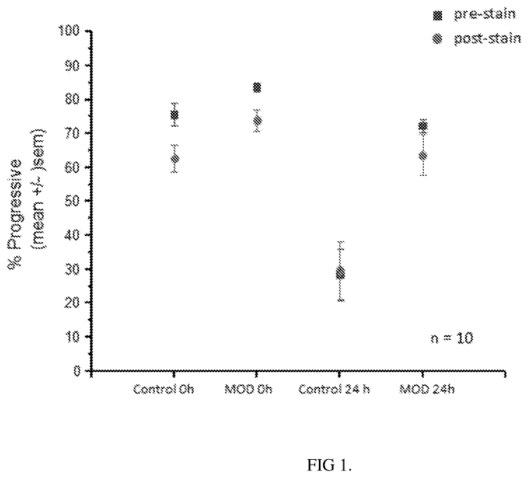

A formulation according to an embodiment of the present invention—CEP2 formulation with additional supplementation (SVFEM)—exhibits enhanced sperm cell survivability and motility compared to commercially available extenders: Andromed® (Minitube, Delavan, Wis. USA) and OptiXell (IMV technologies, Maple Grove, Minn. USA), and a previously described media formulation CEP2 (Verberckmoes et al., 2004), and a. Preliminary data demonstrated the commercially available extenders maintained higher survivability than un-extended ejaculates after 24 hours' incubation, but the proprietary media, SVFEM, was specifically maintained motile cell population within ten percentage points of incoming ejaculate values (FIG. 1). Ejaculates extended with each of the three media options were then stained and run on the sexing cytometers. Andromed® interfered with the ability to distinguish X and Y sperm populations in over 50% of the samples, however, and OptiXell did not maintain a high enough viable population (data not shown). The SVFEM treated group had cell population separation of adequate magnitude to allow proper sexing on the sexing instrument.

In one aspect, media formulations of the present invention include a buffer. For applications where cell survivability and/or shelf life is of preeminent concern, the buffer may be TRIS or HEPES. TRIS is a component of other media commonly used in sperm cell sample production, but the stable pH range for HEPES is closer to the pH of CEP2. In certain embodiments, TRIS may be used due to its longer shelf life in the formulation as measured by pH stability.

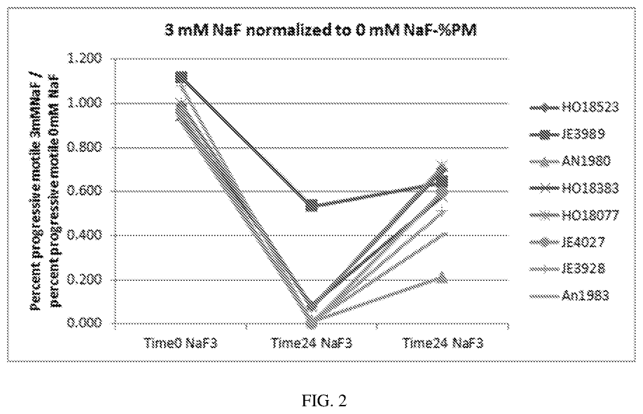

In other aspects, media formulations of the present inventions may be supplemented with NaF was also tested, tested doses of NaF ranged from about OmM to about 6 mM. NaF may be included as a spermatozoa immobilizer, which can conserve cellular energy, and the cellular motility effects of which can be rescued through dilution. However, NaF may decrease the number of motile cells that survived the stresses of the production procedures after packaging and freezing at an equal progressively motile cellular concentration to other tested extender groups (FIG. 2).

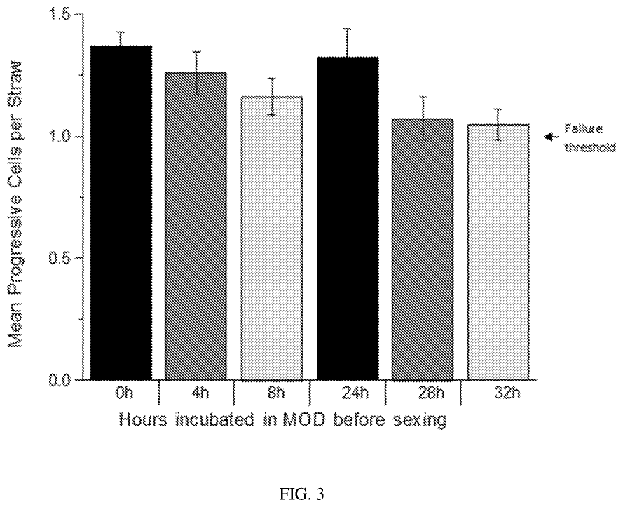

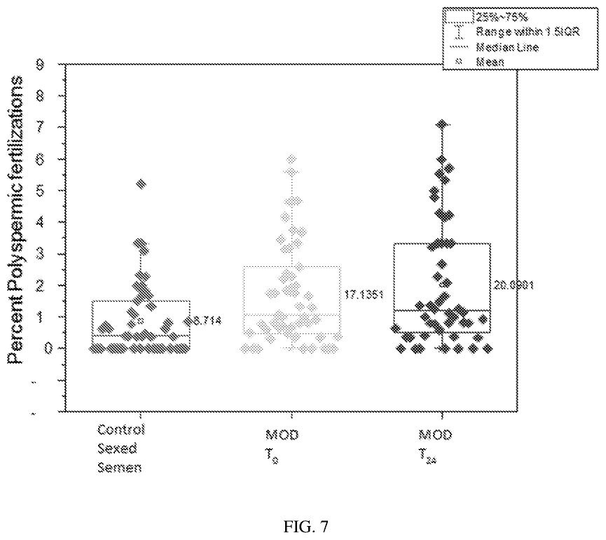

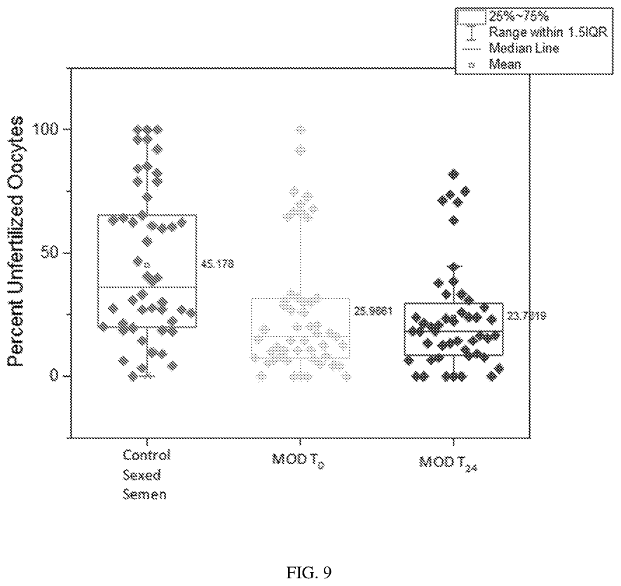

The enhanced media formulation according to an aspect of the invention extended the window of cell survivability before sexing, while still maintaining cells measured as live and motile after the sexing process. As shown in FIG. 3, the number of progressive motile cells after freeze-thaw for SVFEM treated sperm cells passes quality control metric for the concentration of motile cells (1 million progressive motile cells/mL) even after a 24-hour incubation in SVFEM extender before sexing. Quantified motility outcomes were compared among three groups: the non-extended semen sexed same day, SVFEM extended semen sexed same day (T₀ SVFEM), and SVFEM extended semen sexed after 24-hour incubation at 19° C. (T₂₄ SVFEM). For polyspermic fertilizations, the enhanced media formulations according to an aspect of the invention results in a significant increase of presumptive zygotes scored in T₂₄ SVFEM (FIG. 7). Furthermore, as shown in FIG. 9, the percent of unfertilized oocytes calculated out of the total number of presumptive zygotes scored show a significant decrease in both SVFEM treated groups.

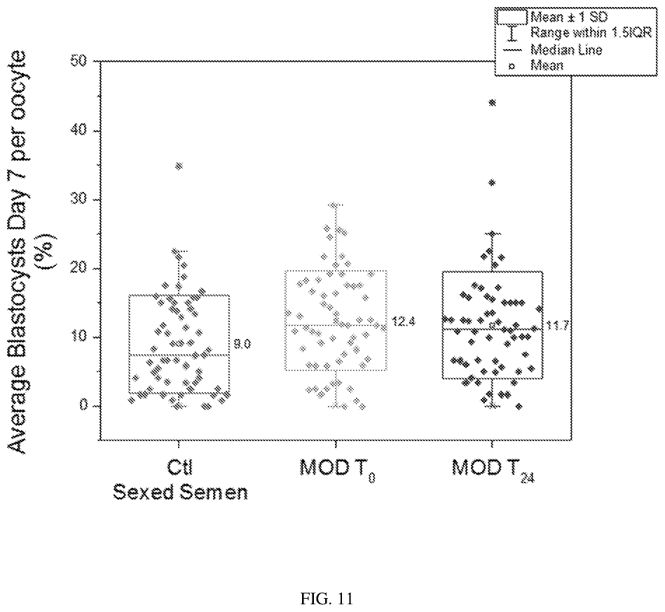

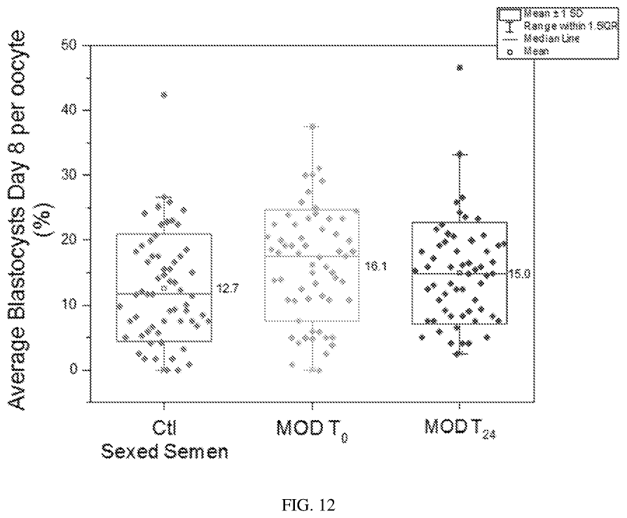

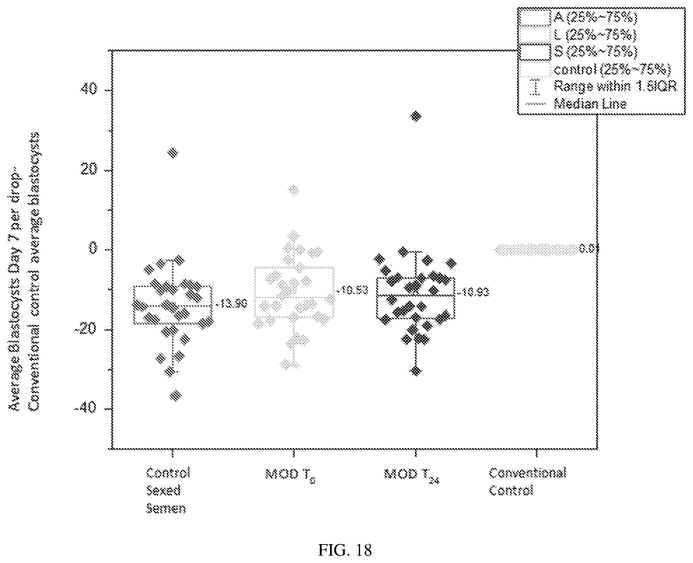

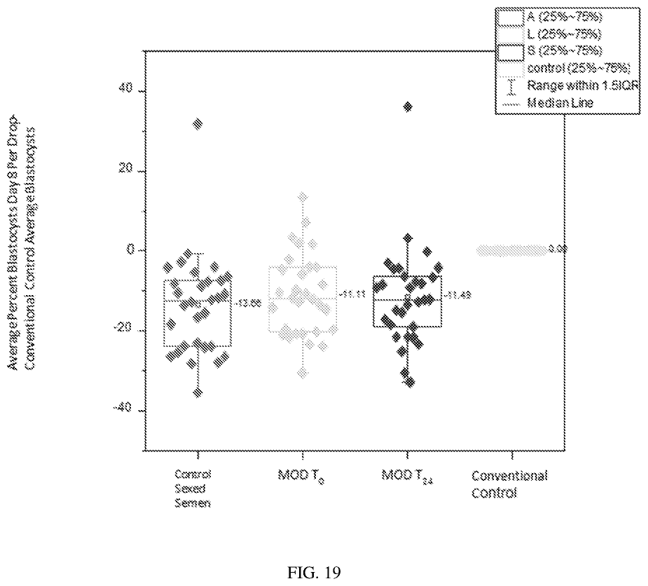

The enhanced media formulations according to an aspect of the invention results in an increase of the percent of blastocysts per oocyte. As show in FIG. 11, the average percent of blastocysts day 7 shows significant increases in the percent of blastocysts per oocyte in both SVFEM and, as shown in FIG. 12, the average percent of blastocysts day 8 shows an increase in the percent of blastocysts per oocyte in T₀ SVFEM group. Blatocyst conversion on day 7 and day 8 for both SVFEM groups is significantly less than the conventional control semen (FIGS. 18 and 19.)

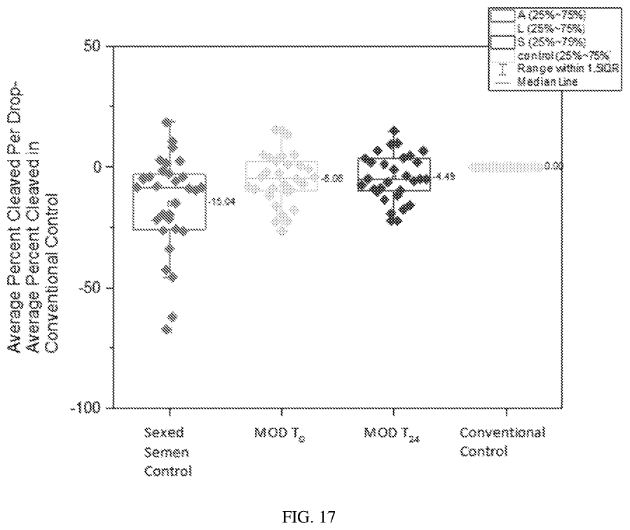

The enhanced media formulations according to an aspect of the invention results in less cleavage. As shown in FIG. 17, both SVFEM groups cleave significantly less than the conventional control.

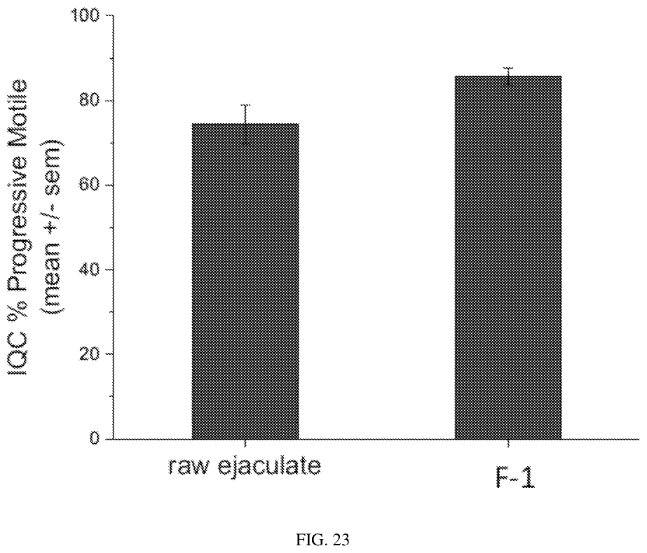

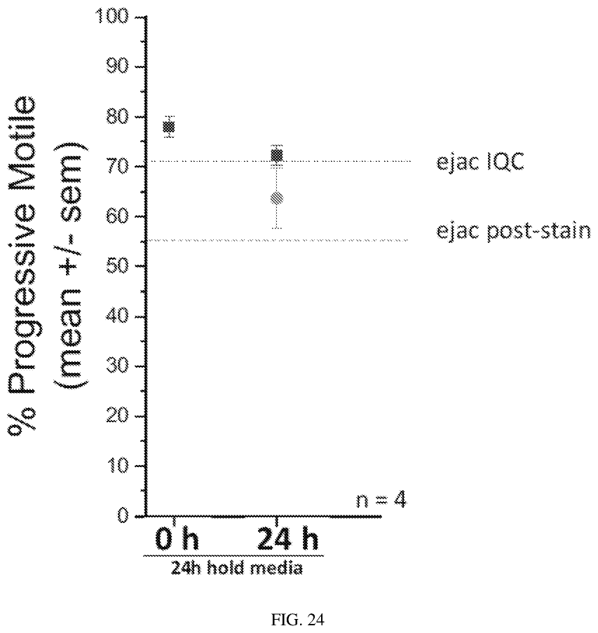

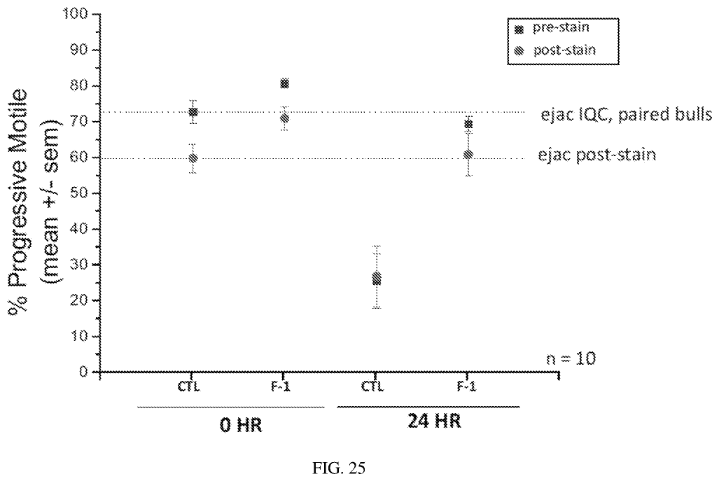

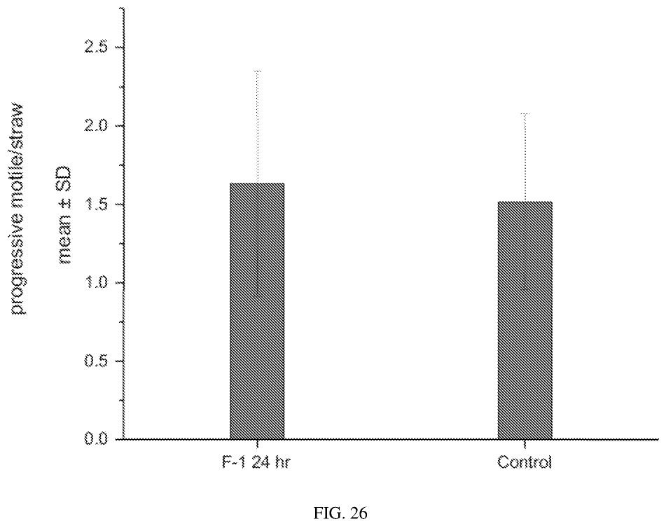

SVFEM extender successfully maintains the motile, viable sperm population for 24 hours before sexing, and results in frozen-thawed sexed semen that meets quality control standards with no increased risk for batch failure compared to current standard operating procedures (for example, as shown in as shown in FIGS. 23-26.) Use of the media, therefore, has the potential to increase utilization of the total ejaculate volume and concurrently increase the number of insemination doses produced per ejaculate, increasing the availability of sexed semen for farmers.

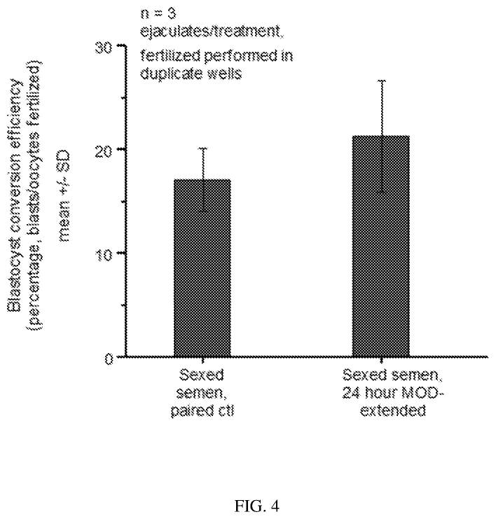

Media formulations according to the present invention maintain sperm in a fertilization competent state. Fertilization competence includes, but is not limited to, the capability of sperm cells exposed to media formulations according to the present invention for producing pregnancies via artificial insemination, and fertilization, cleavage, and blastocyst conversion both in vitro and in vivo. SVFEM treated ejaculates have exhibited fertilization competency, in IVF trials performed with split ejaculates from 20 bulls, collected three times each during the trials. As shown in FIG. 4, this conclusively demonstrated SVFEM extension significantly impacts blastocyst formation for sexed semen. Motile cell numbers indicated no difference in cell survival after freezing compared to the control sexed semen, and IVF assessment of 3 paired ejaculates suggested similar blastocyst conversion between the control and SVFEM extended ejaculates.

In one embodiment, media formulations according to the present invention include antioxidants, which were specifically added to decrease the amount of stress the cells are subjected to during the sexing process. All sperm are exposed to UV light during the sexing process, which typically causes oxidative damage to DNA (rather than direct strand breaks), and sperm is likely subject to elevated reactive oxygen species (ROS) during the cryoprotectant step (Aitken et al., 2015; Farber, 1994). ROS can also cause DNA damage such as single and double strand breaks, and base pair modification (Richter et al., 1988). Fatehi et al. (2006) reported oocytes fertilized with DNA damaged bovine spermatozoa exhibited cleavage rates similar to controls, but further development halted in the damaged experimental group. DNA damage is mitigated by media formulations of the present invention, as shown by similar cleavage rates by higher blastocyst conversion by sperm cells treated with SVFEM compared to control sexed semen produced embryos. This indicates a higher degree of DNA damage in the non-extended semen group. Given the parallel observations, SVFEM extender mitigates DNA damage caused by sexing.

Sperm cells treated with media formulation of the present invention produce more blastocysts per oocyte compared to non-extended, paired controls. SVFEM extended and sexed same day (T₀), and SVFEM extended for 24 hours before sexing (T₂₄) (60 ejaculates total representing 20 bulls from 3 breeds) meet commercial requirements for sexed semen quality. Frozen-thawed sperm from each treatment per ejaculate was tested for their ability to penetrate oocytes, cleave, and produce blastocysts as paired samples. Mono- and poly-spermic fertilized oocytes were quantified via fluorescent microscopy. Embryo development was assessed visually and compared among the treatment groups to identify any differences caused by SVFEM extension.

Sperm treated with media according to the present invention exhibit less DNA damage than non-extended controls. DNA damage was quantified in frozen-thawed sexed semen from the three treatment groups SVFEM T₀, SVFEM T₂₄, and Control using a modified comet assay that specifically detects oxidized base pairs.

Media formulations of the present invention provide beneficial effects for IVF outcomes, measured as cleavage and blastocyst conversion rates measured on day 7 and day 8 post-fertilization. These beneficial effects may be due, at least in part, to mitigation of DNA damage caused by sexing.

Media formulations of the present invention also allow for increased run time for each ejaculate and thereby increase frozen sexed semen product per volume of ejaculate collected and decreased the cost of each insemination dose. This allows sexed semen products to be more widely available to farmers who would profit from the use of sexed semen on their cattle farms. Further the extender is applicable not only to frozen sexed bovine semen, but it could also have applications in extending the life of a fresh ejaculate in a setting where extended transport times are required or specifically for preservation of ejaculates of impaired quality.

The collection and use of sperm cells is a central part of animal husbandry and breeding. Sperm cells are collected in the form of raw ejaculate from male animals and must be stored before further use. Storage can comprise hours or even days. Additionally, cell samples are usually manipulated in one or more ways before use. Thus it is important to maintain the viability and function of the cells throughout the process.

The inventors have developed a medium that maximizes recovery and packaging of functional, fertilization competent sperm. This can improve the flexibility and efficiency of production by exhausting ejaculates, optimizing bull changes, and decreasing the need for backup ejaculates. An additional advantage is an increase in motile cells recovered post-processing (e.g., sexing). When semen samples are processed using the inventive media, increased activity is seen. Increased activity can be increased viability, increased motility or both. Moreover, use of the inventive media allows for greater yields of semen samples after processing.

Other advantages realized by use of the inventive media are a decreased need for multiple collections of ejaculates or moving of animals to the process site. Additionally, the inventive media, due to its maintenance of viability and motility of reproductive cells, can allow for shipping of ejaculates for further processing (e.g., sexing). This eliminates the need to move and quarantine animals.

Processing of raw ejaculate can include many downstream applications, including, but not limited to sorting, sexing (selecting X-chromosome bearing or Y-chromosome bearing cells), freezing, artificial insemination, and IVF (with and without sexing). In some embodiments, this can include cooling and transport of samples, concentrating sperm cells and suspending before staining/sexing.

Sources of reproductive cell samples are typically from ejaculate, obtained by methods commonly known in the art. The ejaculate samples can be a single source or pooled. In some embodiments, in vitro produced or expanded sperm cell populations are contemplated. Samples are obtained from animals, preferably mammalian animals; more preferably livestock; samples are most preferably porcine or bovine.

In some embodiments, the composition is utilized as a “hold media” to store raw ejaculate and minimize loss of reproductive cell components. In other embodiments, the composition functions as a medium to use for processing of reproductive cell samples that are used for further processing (such as, e.g., sexing). In further embodiments, the composition is utilized as a “hold media” to store isolated sperm cells after processing and before use in breeding procedures. This can also be referred to as an “extender media” since samples remain viable for longer when the inventive media is used.

In certain embodiments, compositions comprising reproductive cells and the inventive “hold media” maintained acceptable viability and/or motility for hours; in particular embodiments, the extension was for 24 hours. In other embodiments, the inventive compositions maintained an acceptable level of live cells throughout cell processing; in particular embodiments, the percentage of dead cells were ≤25% throughout sexing duration of processing.

Samples can be combined with the improved media in a variety of ways. The media can be added directly after collecting the raw ejaculate sample, within a set amount of time after collecting the raw ejaculate; or the raw ejaculate can be collected directly into the media.

Sperm cells that are exposed to the inventive media exhibit enhanced motility, viability, and functionality (including the ability to fertilize ova) over time, compared to sperm exposed to existing commonly-used hold media. Thus, these media can enhance yields in both conventional and sexed semen production. The inventive media maximizes recovery and packaging of functional, fertilization competent sperm.

The disclosure relates to compositions that improve reproductive cell viability and activity throughout storage and processing. In one aspect, the compositions comprise a base salt media with at least one additive selected from the group consisting of antioxidants, phosphatidylserine (PS), coumarin compounds or pyranocoumarin compounds, zinc chloride, coenzyme Q10, a nonsteroidal anti-inflammatory drug (NSAID), linolenic acid, fatty acids, D-aspartic acid, and combinations thereof. In some aspects, the compositions contain sodium fluoride. These compositions serve as improved media for storage and processing of reproductive cells.

In some aspects, the compositions comprise Nonsteroidal Anti-inflammatory Drugs (NSAIDs), which are a class of drugs and compounds capable of reducing inflammation, primarily through inhibition of cyclooxygenase enzymes (COX-1 and/or COX-2). The compositions can include one or more NSAID including, but not limited to: salicylates, including aspirin (acetylsalicylic acid), diflunisal (Dolobid); salicylic acid and other salicylates, and salsalate (Disalcid); Propionic acid derivatives, including Ibuprofen, Dexibuprofen, Naproxen, Fenoprofen, Ketoprofen, Dexketoprofen, Flurbiprofen, Oxaprozin, and Loxoprofen; acetic acid derivatives, including indomethacin, Tolmetin, Sulindac, Etodolac, Ketorolac, Diclofenac, Aceclofenac, and Nabumetone; enolic acid (Oxicam) derivatives, including Piroxicam, Meloxicam, tenoxicam, Droxicam, Lornoxicam, Isoxicam, and phenylbutazone (Bute); anthranilic acid derivatives (Fenamates), including mefenamic acid, meclofenamic acid, flufenamic acid, and tolfenamic acid; selective COX-2 inhibitors (Coxibs), including Celecoxib, Rofecoxib, Valdecoxib, Parecoxib, Lumiracoxib, Etoricoxib, and Firocoxib; Sulfonanilides, including Nimesulide; and other NSAIDs, including Clonixin, Licofelone, and H-harpagide (in Figwort or Devil's Claw). In some embodiments, the compositions comprise coumarin compounds or pyranocoumarin compounds. In certain embodiments, the coumarin compound or pyranocoumarin compound comprises decursin.

In some embodiments, the base salt media is synthetic cauda epididymal plasma (CEP2), which is described in the literature (1). This preparation contains sodium chloride (NaCl), potassium chloride (KCl), calcium chloride dehydrate (CaC₁₂(H₂O)₂), magnesium chloride hexahydrate (MgC₁₂(H₂O)₆), sodium bicarbonate (NaHCO₃), sodium phosphate dihydrate (NaH₂PO₄(H₂O)₂), potassium phosphate (KH₂PO₄), fructose, sorbitol, Bovine Serum Albumin (BSA), TRIS base and citric acid. In one embodiment, the inventive media contains CEP2 as the base salt media and the additives of phosphatidylserine (PS), decursin, zinc chloride, coenzyme Q10, acetylsalicylic acid (aspirin), linolenic acid, fatty acids, D-aspartic acid, and sodium fluoride.

Other base salt media are contemplated for use in creating the inventive media. In one embodiment, Tyrode's albumin lactate pyruvate (TALP) is contemplated. Tyrode's is an isotonic solution preparation containing sodium chloride (NaCl), potassium chloride (KCl), disodium phosphate (Na₂HPO₄), sodium bicarbonate (NaHCO₃) and magnesium chloride hexahydrate (MgCl₂(H₂O)₆. In one embodiment, the pH is about 6.6-6.8.

The base salt media has additives therein to bring about the desired properties and create the inventive media. In one aspect, the additives comprise one or more of phosphatidylserine (PS), decursin, zinc chloride, coenzyme Q10, acetylsalicylic acid (aspirin), linolenic acid, fatty acids, D-aspartic acid, sodium fluoride, and combinations thereof. In some embodiments, all of the additives are included in the formulation. In other embodiments, one or more of decursin, zinc chloride, coenzyme Q10, acetylsalicylic acid (aspirin), linolenic acid, fatty acids, D-aspartic acid, or sodium fluoride are omitted. Concentration ranges for the additive ingredients are shown in Table 1.

TABLE 1
Concentration ranges for additive ingredients

| Component | Approx conc range | |
|---|---|---|
| phosphatidylserine | 0-5 mM (2 mg/mL) | |
| decursin | 0-10 | uM |
| zinc chloride | 0-10 | ug/mL |
| coenzyme Q10 | 0-50 | ug/mL |
| aspirin | 0-1 | mM |
| linolenic acid | 0-5 | ng/mL |
| fatty acid supplement | 0-1 | uL/mL |
| D-aspartic acid | 0-500 | ug/mL |
| NaF | 0-6 | mM |

Specifically, the inventors have discovered that phosphatidylserine (PS) is a key ingredient (FIG. 9), which replaces other phospholipids (e.g., phosphatidylcholine) in commonly-used hold media. Although other phospholipids are contemplated, PS has proved advantageous over others. This was a surprising and unexpected finding. Phosphatidylserine containing media can be difficult to formulate, and there is a general acceptance in the art that phosphatidylserine is not needed or that alternatives to phosphatidylserine are sufficient.

In some aspects, the following compositions contemplated are sperm cell compositions that comprise sperm cells, and one or more of phosphatidylserine (PS), decursin, zinc chloride, coenzyme Q10, acetylsalicylic acid (aspirin), linolenic acid, fatty acids, D-aspartic acid, sodium fluoride, and combinations thereof. The compositions may include seminal fluid components.

In other aspects, a container of sperm cells is contemplated which comprises a plurality of sperm cells, a base salt media, and one or more of phosphatidylserine (PS), decursin, zinc chloride, coenzyme Q10, acetylsalicylic acid (aspirin), linolenic acid, fatty acids, D-aspartic acid, sodium fluoride, and combinations thereof. The container may further comprise seminal fluid components. Such containers can be used for storage, or for further procedures such as IVF or AI.

In other aspects, compositions that produce enhanced zygote/blastocyst formation from germ cells (i.e., increased fertilization or increased activity of reproductive cells) are contemplated. In one aspect, the compositions comprise a base salt media with one or more of phosphatidylserine (PS), decursin, zinc chloride, coenzyme Q10, acetylsalicylic acid (aspirin), linolenic acid, fatty acids, D-aspartic acid, and combinations thereof. In some aspects, the compositions also contain sodium fluoride. In such compositions, reproductive cell activity of stored and/or manipulated samples is maintained or even increased, by the measure of either motility, fertilization, or both. Fertilization can be measured by blastocyst formation. The inventors have found that the inventive media improves sexed semen fertilization rates in IVF and/or AI

In vitro fertilization can be carried out by methods and procedures known in the art. Many factors can affect successful IVF, including, but not limited to sources of eggs; sperm samples and additional processing/manipulation; fertilization, presence/concentration of media components/sperm cells during fertilization step; and presence/concentration of media components during blastocyst formation/embryo development.

Media is combined with cells in a variety of ways, for example by using a set volume of media; a set ratio of media to sample, or media provided at a set volume in relation to a measured aspect of the sample (i.e., sperm cell concentration). In certain aspects, media is added to the sample, in other embodiments, the sample is added to the media. In other embodiments, both sample and media are added to a third container/receptacle.

Some aspects and embodiments of the disclosure are illustrated by the following examples. These examples are provided to describe specific embodiments of the technology and do not limit the scope of the disclosure. It will be understood by those skilled in the art that the full scope of the disclosure is defined by the claims appending this specification, and any alterations, modifications, or equivalents of those claims.

### Example 1

Sexing procedures require that spermatozoa survive a multitude of insults, and while preliminary data demonstrated that SVFEM extender facilitates successful sexing up to 36 hours post-collection, the fertilization capacity of the extended sperm had yet to be evaluated. While scientists have attempted to correlate in vitro sperm characteristics with fertilization outcomes, no such assay has become a gold standard. Cellular assessments of the plasma membrane, intracellular structures, as well as tests of velocity parameters with a CASA system or with the bovine cervical mucus penetration test (BCMPT) often have conflicting results in how they relate to IVF or A fertility. For example, PI staining of plasma membrane integrity correlating with field fertility for Januskauskas et al. (2003) and no correlation for Oliveira et al. (2012). Also, distance traveled in BCMPT appeared to correlate to NRR (Tas et al., 2007; Bacinoglu et al., 2007) but that this assay did not relate well to IVF outcomes (Keel and Schalue, 2009). These conflicting results prevent relating sperm viability or motility measurements to performance in IVF or AI. Accurately assessing spermatozoa fertility, therefore, requires performing an IVF or AI.

While an AI trial is required to apply the SVFEM extension to commercial products, an IVF trial quantifies cleavage and blastocyst conversion rates, providing insight into mechanisms underlying apparent changes in the number of embryos produced (Bermejo-Alvarez et al., 2010; Blondin et al., 2009, Greve and Madison, 1991). Quantified outcomes in this IVF trial will include percent fertilization, cleavage conversion, and blastocyst conversion rates day 7 and day 8. These two time points to quantify blastocyst development are based on literature showing a developmental delay in IVF using sexed semen (Lu et al., 1999), and will indicate whether SVFEM extension changes the rate of embryonic development.

While correlations between IVF and AI performance are not confirmed (Holden et al., 2016), an IVF trial is also a lower risk assessment of early embryonic timepoints because it does not involve impregnating the animal which can be detrimental to the animal's health or can have impacts on the farm financially if no viable pregnancy results. If this trial is successful it would then also justify further analysis of actual pregnancy and calving rates through an AI field trial or embryo transfer of IVF produces blastocysts.

Based on the data of velocities measured post freezing after incubation with SVFEM and the preliminary IVF assessment with three paired ejaculates in FIG. 4, it is hypothesized that extension with SVFEM media will perform as well as unextended ejaculates. The ability of the SVFEM extended ejaculates to perform as well as the non-extended ejaculates in IVF would verify that SVFEM extender could be used in a sexed semen product and still allow for fertilization and early embryonic development. This increases the time available to sex the ejaculate volume and would allow for an increased number of sexed insemination doses produced per volume of ejaculate collected. This increase in production would improve farmer access to these genetically verified sex-skewed insemination doses.

Materials and Methods:

Media formulas are outlined below.

IVF Testable Unit Generation Design: This study utilizes sires from three breeds, Holstein, Jersey, and Angus. The study design included three ejaculate collections from 20 unique sires to generate sexed semen units to prevent a single bull's variation from skewing the data significantly. Prospective power calculations using the preliminary data indicated the triplicate collections from 20 bulls would provide a statistical power of 0.9 or greater for the blastocyst conversion outcomes from the split ejaculate design including two SVFEM treatments and the paired control.

Final breed tally of the collections were 16 Holsteins, 2 Jersey, and 3 Angus sires. These bulls ranged in age from 1.5 to 9.5 years old during unit collection. American Breeders Service provides a field fertility (natural service) star ranking from 1-5 stars such that the best-calculated fertility is presented as a 5-star ranked bull. Ejaculates collected during this trial were from sires from the full range of star ranking, from unranked to 5 stars. This allows for investigation of how SVFEM extender impacts ejaculates from high and low field fertility ranked sires, and if it affects sires of different age or breed differentially.

Ejaculates selected for use in this trial were processed following standard production procedures outlined in detail below. Insemination doses, freeze canes, and all associated documentation were blind labeled during the incoming quality check. This was to prevent technician bias on quality control assessments after freeze-thaw as well as during the IVF outcome quantitation assessments.

After processing, the sexed semen was packaged and frozen during a regularly scheduled production freeze following standard SOPs. Within one week of unit generation, the outgoing quality control measurements were performed by a trained QC technician. Post-thaw motile concentration and the presence/absence of bacterial contamination were completed. Insemination doses had to pass standard production outgoing quality control parameters to be utilized for the IVF trial.

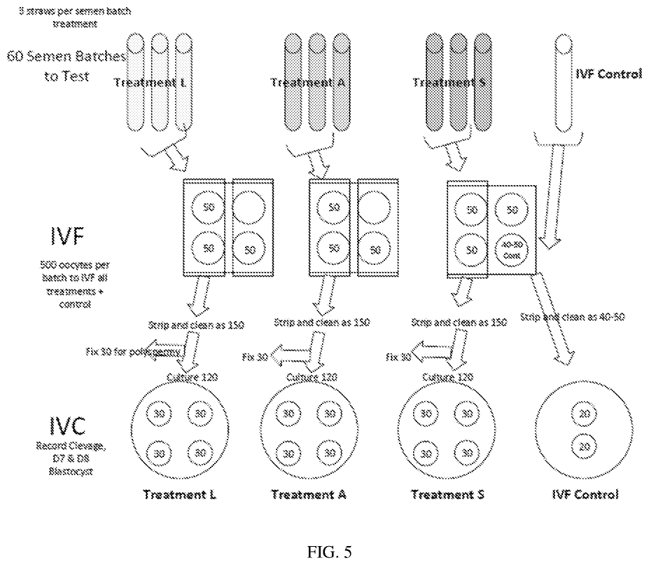

IVF Trial: IVF was performed by two different facilities. To prevent inter-facility differences during IVF trials, each facility tested their separate IVF protocols in parallel as they differed. After testing fertilization and maturation in multi-well plates and in droplets under mineral oil, an optimal protocol was identified. Insemination doses from the same ejaculate were used for IVF at both facilities following the same protocol and cleavage and blastocyst conversion rates were compared. Once the protocol was verified at both facilities and equitable blastocyst conversion rates between the facilities were achieved, testing was conducted on the SVFEM IVF produced insemination doses progress. The protocol is outlined visually in FIG. 5.

Each facility received the three treatment groups for each individual ejaculate. The three treatments from each split ejaculate were used concurrently to fertilize oocytes from the same pooled batch of slaughterhouse oocytes. A conventional non-sexed control straw, with previously verified fertilization capacity, was also concurrently run to verify the viability of the oocytes. To ensure that no false failure of development was due to poor oocyte quality, IVF reactions for an ejaculate were repeated if the conventional control group also failed to develop at approximately 15% blastocyst conversion, which indicates an oocyte quality issue.

The trained IVF technicians at both facilities performed the outlined fertilizations. Blastocysts were scored on days 7 and 8 post-fertilization, then collected and fixed. To prevent inadequate conclusions due to high variance, an ejaculate was repeated if the variance among the technical replicates was above 20%.

Ejaculate Collections: All ejaculate collections were performed on-site by experienced technicians following standard collection procedures.

Ejaculate extension and Incoming Quality Assessment: The volume of the ejaculate was determined using a serological pipette and evenly divided into two tubes. Immediately, within 15 minutes, the SVFEM extender was added in a 1:1 ratio to the SVFEM extended half of the split ejaculate. They were then transported in an insulated cooler to prevent temperature fluctuation to the second facility. On arrival, GTLS antibiotic solution was added at a 2% v/v of ejaculate.

Within 45 minutes of initial ejaculate collection, the cell concentration and incoming motility parameters were collected. Concentration was determined using a Nucleocounter SP-100™ with Reagent S100 and SP100 cassettes (ChemoMetec Allerod, Denmark). Motility characteristics were calculated by diluting 10 μL sample in 990 μL motility diluent and reading 7 frames each in 2 chambers of Leja 4 chamber capillary slides with a known chamber depth on a Hamilton Thorne IVOS II using HTCasa II software at 60 Hz frame capture speed in a 37° C. enclosed stage with a Zeiss 1× objective. An ejaculate was utilized if the cell concentration was greater than 500 million/mL, and the percent of progressive motile cells in the sample was ≥65%.

Sample Preparation for Sperm Sexing: A stained sample was prepared at room temperature that contained 200 M/mL sperm cells in 0.06 mg/mL Hoechst 33342 diluted to final volume in Stain TALP. The sample was then incubated in a 37° C. water bath for 45 minutes. After 45 minutes Red Stain TALP was added to the stained sample in a 2:1 v/v ratio. The sample +Red TALP was then thoroughly mixed using inversion, filtered using tube top 20 μm Partec filters (Partec #04-0042-2315), and aliquoted into round bottom 5 mL tubes.

Sexing Cytometer Metrics: The stained, filtered sample was then run on proprietary sexing cytometers. The sample throughput was adjusted to 17,500 cells/sec and the detection and kill lasers were focused. To confirm proper laser focus, kill count assessments were performed before collecting sex skewed sample. A successful kill count has a population that is ≥75% dead and ≥95% sliced withat least 200 cells being counted. If an instrument could not achieve the above metrics, the instrument was not used to collect sex skewed semen. After a successful kill count, a gate was placed to collect the X chromosome cells, which is the cell population with the brighter Hoechst 33342 fluorescence as measured with a 355 nm wavelength excitation laser. Cytometer performance metrics were collected 15 minutes after instrument set up, and 15 minutes after the placement of the last sample collection tube, including the height of the Y-peak, the height of the X-peak, the height of the trough from the histogram of events per emitted fluorescent intensity, gated %, and dead %.

Sex Skewed Sample Collection and Processing: The sample was run to collect between 300 and 400 mLs of sex skewed sample, the composition of which is approximately 17% TRIS A buffer, 80% sheath fluid, and 2% cell sample. The sample was collected in 50 mL conical tubes containing 5 mLs of TRIS A, and each tube was filled to a max volume of 30 mLs before being replaced. After the requisite total volume was collected, sexed sperm was centrifuged at room temperature at 2400×g for 10 minutes. The supernatant was aspirated and discarded to reach a 1 mL pellet volume. 17 μL of GTLS antibiotic solution was added to each pellet after resuspension. The tubes were then placed in beakers filled with 150 mLs of room temperature water, to prevent cold shock, and were then transferred to a 4° C. cold room.

After 90 minutes of equilibration at 4° C. the samples were cryoprotected by adding TRIS B (which contains glycerol) to a final concentration of 20% v/v of sample in 3 separate additions, 15 minutes apart. The concentration of progressively motile cells was determined using the Hamilton Thorne IVOS II with HTCasa Animal Breeders II software set to the same capture settings listed above, but with a Xenon light source and an Olympus 10× UplanSApo objective. The cryoprotected sample was diluted to final live, motile cell concentration, 2.5 M/mL, in Packaging Extender and placed in Mini Straws which hold 0.25 mL volume (IMV technologies, Maple Grove, Minn. USA) using an MX4 straw filling and sealing machine (IMV technologies, Maple Grove, Minn. USA). Filled straws were rapidly cooled using a freeze tunnel before storage in liquid nitrogen.

Outgoing Quality Control Assessment: To assess the number of motile cells that survived the freezing process a single straw is thawed in a 37° C. water bath for 45 seconds. The straw is then plunged into a pre-warmed Eppendorf tube. The sample is then gently vortexed 10 seconds to homogenize the sample. After which 20 μLs of sample is added to 20 μLs of QC diluent, gently vortexed for 5 seconds, and read on the CASA fluorescent settings described above. The remainder of the straw volume is spread on a blood agar plate and left at 37° C. for 24 hours before bacterial colonies are counted.

In Vitro Fertilization and Assessment:

Oocyte prep: Four well fertilization plates were prepared by filling all 4 wells with 400 μL of BO-IVF (MOFA Verona, Wis.) and equilibrated in a 37° C. 5% CO₂ for at least 1 hour. At this same time, four well embryo culture plates filled with 450 μL of BO-IVC (MOFA Verona, Wis.) were made and equilibrated at 37° C. 5% CO₂, 5% O₂. A sample of each lot of BO-IVC used during these fertilizations was aliquoted and stored at −80° C. as control media for assessing conditioned embryo media. All handling of oocytes and zygotes was done with heat pulled glass pipettes.

Cumulus oocyte complexes (COCs) were collected from slaughterhouse ovaries by aspiration. The COCs were kept warm in oocyte maturation media (MOFA Verona, Wis.) and handled on 37° C. heated stages. COCs were grouped and separated into 3 wells of 60 oocytes each per treatment group in a 4 well plate.

Semen prep: Three insemination straws per treatment group were thawed at 37° C. for 45 seconds. They were then layered over 80% BoviPurem density gradient (Nidacon international AB. Sweden). The samples were centrifuged at 500×g for 15 minutes, aspirated close to the pellet, and then resuspended in warm TL HEPES (MOFA Verona, Wis.). They were centrifuged at 300×g for 5 minutes, aspirated to 100 μLs, and the pellet was resuspended in that low volume. A 5 μL sample aliquot was added to 95 μLs 4% NaCl to immobilize the cells, and cell concentration was quantified using a hemocytometer. Cells with visible membrane damage were not counted towards cell density calculations. Sperm suspension was added to the COC containing wells at 1.2 million sperm per well (20,000 sperm/oocyte).

Cumulus oocyte complex removal: 24 hours after sperm addition, COCs from the same treatment group were pooled in a 15-mL conical tube containing 0.5 mL TL HEPES with 1 mg/mL hyaluronidase. COCs were vortexed for 1 minute, put back into the 37° C. heating block for 1 minute, and vortexed again for 1 minute. The presumptive zygotes were washed in TL HEPES plates and then placed in the embryo culture plates containing maturation media (MOFA Verona, Wis.) that were equilibrated for 24 hours prior to use (oocyte prep above). The presumptive zygote containing plates were then placed at 37° C. 5% CO₂, 5% O₂ for the rest of the IVF trial.

Development assessments: Developmental assessments were performed three times during the 8-day post-fertilization incubation. Cleavage events were quantified 48 hours after initial fertilization. Blastocysts were scored on a binary scale of yes/no blastocyst based on its developmental stage. If the embryo had reached at least the early blastocyst stage it was scored as a blastocyst. The differences between early, expanding, and hatched blastocysts were not recorded, nor were the blastocysts scored, but blastocysts were fixed to facilitate future characterization. Blastocyst conversion per oocyte was visually determined on both day 7 and day 8 after initial fertilization. All determinations of developmental stages were done by trained IVF technicians using a dissecting scope on a heated stage set to 37° C.

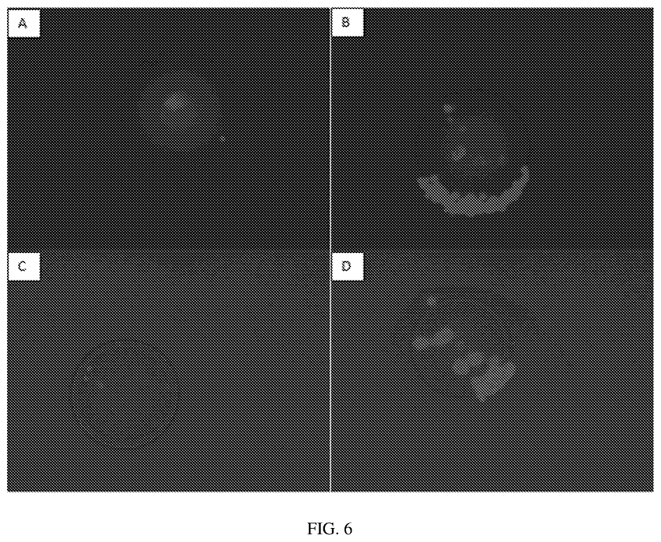

Assessment of early fertilization events: 24 hours after initial fertilization and after the presumptive zygotes were stripped of their COCs, a subset of 20-30 zygotes were fixed and stained using a proprietary kit created to assess for monospermic/polyspermic events. The DNA stain is Hoechst 33342. All presumptive zygotes were scored in 1 of 4 categories: monospermic fertilization, polyspermic fertilization, unfertilized, or other. The other category encompasses zygotes that were present, but un-scorable due to either obscuring fluorescence from COC not fully removed or because the zygote was fragmented. Those zygotes presenting with two pronuclei were considered monospermic, and those presenting with 3 or more were scored as polyspermic (Yang et al., 1993). Images showing representative images for each score are shown in FIG. 6.

Fixing of samples day 8 post-fertilization: After the final assessment of blastocyst conversion on day 8 the samples were fixed in Invitrogen™RNAlater™ (ThermoFisher Waltham, Mass. USA). The blastocysts were divided into two groups and placed into two tubes to allow for repetitive measures on genetic assessments. Degenerated zygotes were added to a third tube. 40 μLs of RNAlater™ per blastocyst or degenerate was added to each tube, 10:1 v/v addition as recommended by ThermoFisher. These samples were placed at 4° C. for at least 24 hours, but no longer than 1 week, before being transferred to −20° C. for long-term storage in accordance with the handling instructions provided by ThermoFisher. Conditioned maturation media from the droplets were also collected and stored at −80° C. to preserve RNA (Vaught and Henderson, 2011).

Statistical Analysis: All statistical analysis was performed using OriginPro 2017 64-bit software. Threshold for significance was set at α=0.05. Transformations of the raw data were performed to normalize the data allowing for ANOVA analysis. Arcsine transformations were adequate to normalize for all data except two: the polyspermic and unfertilized percents, which were transformed using a natural log transformation instead.

Results: The earliest zygotic timepoint assessed during this experimentation was the fertilization efficacy amongst the treatment groups. Fertilization events were quantified as a percentage of the total number of assessed zygotes (initial oocytes) sampled from each treatment well. To achieve normal distribution with the reported raw data, arcsine transformations were performed on all data sets except two, the polyspermic and unfertilized events, which required natural log transformations to achieve normalcy. Normalcy tests were performed in OriginPro (not shown).

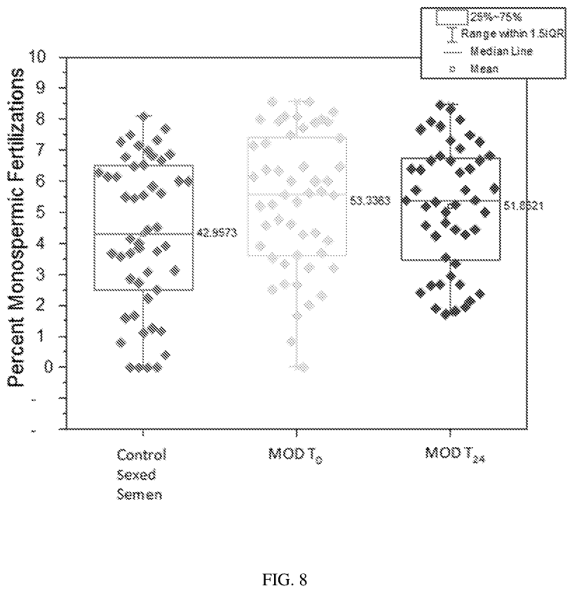

Monospermic events were not significantly different among the groups with the To SVFEM treated group at 55.7% monospermic fertilization, control, 45.3% (Bonferroni means comparison T₀ v. control p=0.240), and T₂₄ SVFEM, 54.3, %, p=0.651 vs. control (FIG. 8). Unfertilized oocytes occurred at significantly lower percentage in SVFEM treated groups compared to control; control=45.2%, T₀ SVFEM=26.0%, and T₂₄ SVFEM=23.7% unfertilized oocytes (FIG. 9, ANOVA p=0.002, Bonferroni means comparison p=0.004 for To SVFEM v. control, and p=0.014 for T₂₄ SVFEM v. control). The percent of polyspermic events appeared elevated in SVFEM-treated samples, with 8.7% polyspermic fertilizations for control, 17.1% for T₀ SVFEM, and 20.0% for T₂₄ SVFEM, but was not significantly different across groups with a one-way ANOVA on natural log-transformed data overall p-value of 0.279 (FIG. 7). These data indicate that SVFEM-treated groups exhibited more total fertilization events, with a potential risk for increasing polyspermic fertilizations under these specific IVF conditions.

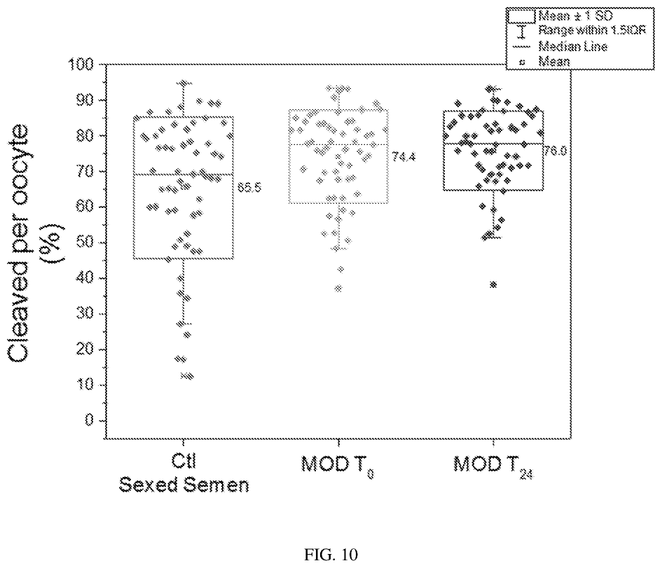

Embryonic developmental was assessed by quantifying cleavage events on day 2 post-fertilization. The percent of cleaved zygotes per oocyte fertilized was 65.5% in control, 74.4% in T₀ SVFEM, and 76.0% in T₂₄ SVFEM (FIG. 10). The percent of cleaved embryos was significantly greater in both treatment groups with Bonferroni p-values of 0.002 for T₀ v. control and 0.001 for T₂₄ v. control, indicating that both SVFEM treated groups, To and T₂, increased the percentage of cleaved embryos per oocyte fertilized.

The percentage of blastocysts per oocyte quantified on day 7 post-fertilization was also arcsine transformed to facilitate ANOVA analysis. Mean percent blastocysts on day 7 were 9.0%, 12.4%, and 11.7% for control, T₀ SVFEM, and T₂₄ SVFEM, respectively (FIG. 11). Both treatment groups were significantly different from control, with Bonferroni means comparisons of p=0.008 for T₀ SVFEM v. control, and p=0.002 for T₂₄ SVFEM v. control. These data demonstrated blastocyst conversion per oocyte fertilized by SVFEM treated spermatozoa increased on day 7 post-fertilization compared to non-extended control sexed semen.

The final assessment of embryo development was performed on day 8 post-fertilization. Mean percent blastocyst per oocyte fertilized were 12.7%, 16.1%, and 15.0% for control, T₀ SVFEM, and T₂₄ SVFEM, respectively (FIG. 12, n=46), with a significant difference between the sexed control and T₀ SVFEM (p=0.037, Bonferroni means comparison). For T₂₄ SVFEM compared to control, p=0.143. Fertilization with SVFEM extended sperm which was sexed same-day increased the number of blastocysts per oocyte fertilized by 3 percentage points, or a 25% increase, as measured on day 8 over the split ejaculated sexed control, and SVFEM extended ejaculates sexed after a 24-hour incubation exhibited blastocyst conversion rates not statistically different from control non-extended sexed semen. Due to the possible increase in monospermic events, a Pearson correlation analysis was performed to determine whether the percent monospermic fertilizations correlated with the number of blastocysts on Day 8. The p-value calculated in the Pearson correlation was <0.001 suggesting a positive relationship exists between these two outcomes.

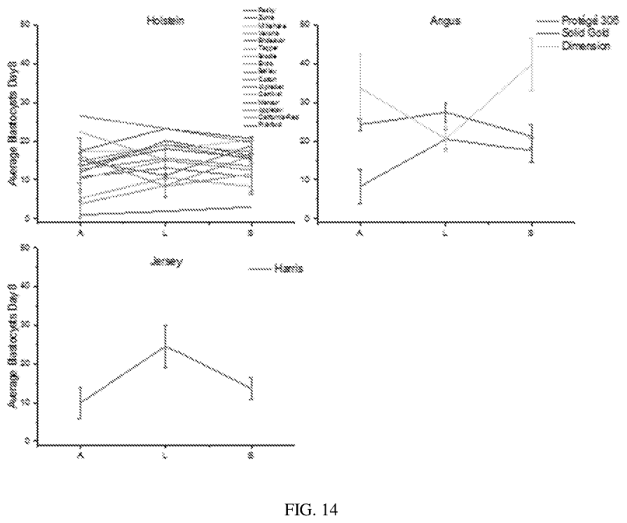

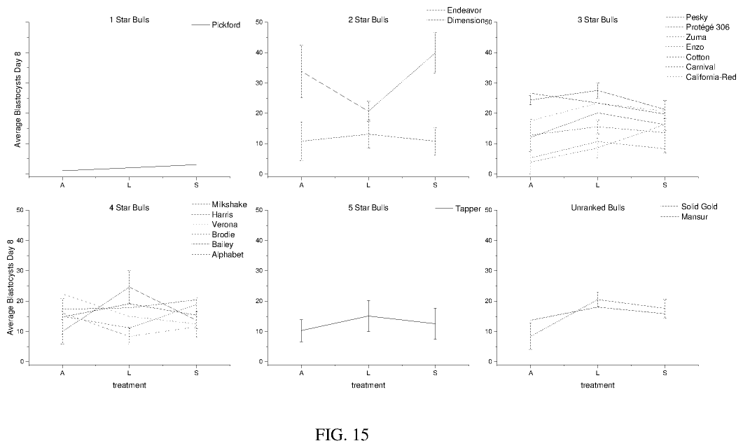

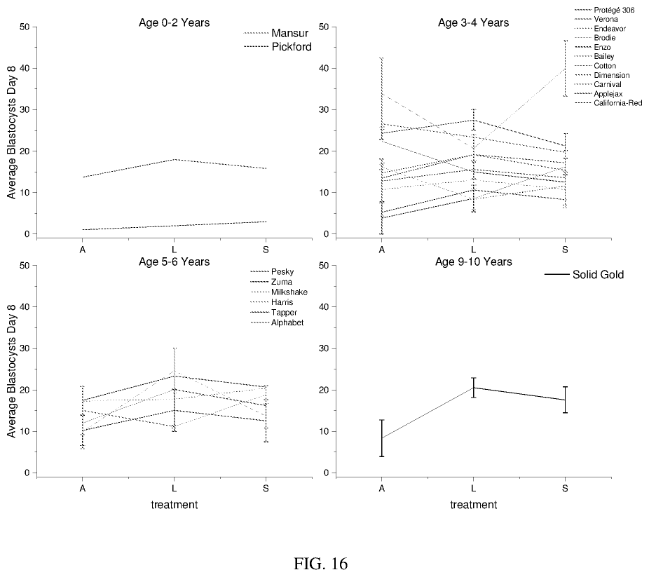

Additional analyses were performed to determine whether differences in blastocyst conversion measured on day 8 could be attributed to breed, star rank, or bull age. The average blastocyst percent per oocyte fertilized on day 8 was calculated for each bull by averaging the outcomes from all collected ejaculates assessed with IVF during this trial. Scatter plots were generated separating the bulls based on the breed in FIG. 14, star rank in FIG. 15, and age range in FIG. 16 do not reveal any corollary trends. Separation into these categories was uneven, and often at least one category was represented by only one sire, limiting the ability to draw statistical conclusions from this analysis.

A second set of analyses compared both the SVFEM treated and sexed semen control to the conventional, non-sexed semen that was used as an oocyte quality control. For all parameters measured during the IVF trial; cleavage, blastocysts per oocyte day 7, and blastocysts per oocyte day 8, the conventional un-sexed control semen was significantly greater than both treatment groups and the sexed semen control (ANOVAs, p<0.001, FIGS. 17, 18, and 19). Conventional semen was not assessed for fertilization efficiency through the monospermic analysis. These data show that while SVFEM-treated ejaculates may increase cleavage or blastocyst conversion rates compared to the control sexed semen, the semen extender did not increase bring the sperm fertilization performance to a level comparable to un-sexed, conventional semen.

Discussion: The results outlined above indicate that SVFEM-treated ejaculates produce fertilization competent spermatozoa, demonstrated by monospermic fertilization events occurring at a statistically similar percent in both SVFEM ejaculates compared to non-extended controls. These fertilized zygotes are also capable of undergoing embryo development past the point of the activation of the embryonic transcript at about the 8-cell stage (Viuff et al., 1996) and to the morphologically assessed blastocyst stage. The number of monospermic fertilizations is positively correlated with the average number of blastocysts counted on day 8 in a Pearson's Correlation Coefficient test with a p-value <0.001, suggesting that this increase in blastocyst conversion on day 8 could be due to the increase in monospermic fertilization.

The incidence of polyspermy does not appear to exceed expected levels based on historical publications for any of the tested groups. Previous work quantified the polyspermic incidence in mature oocytes in bovine IVF using un-sexed semen at about 15% (Cebrian-Serrano et al., 2012). None of the groups, control, T₀ SVFEM, or T₂₄ SVFEM, demonstrated polyspermic fertilization occurrence significantly greater than the literature-reported 15% for conventional un-sexed semen (one-sided t-test) The conventional unsexed semen fertilized zygotes produced were not assessed with pronuclear staining, however, so a direct comparison to the number of polyspermic events under specific testing conditions with un-sexed bovine semen cannot be drawn.

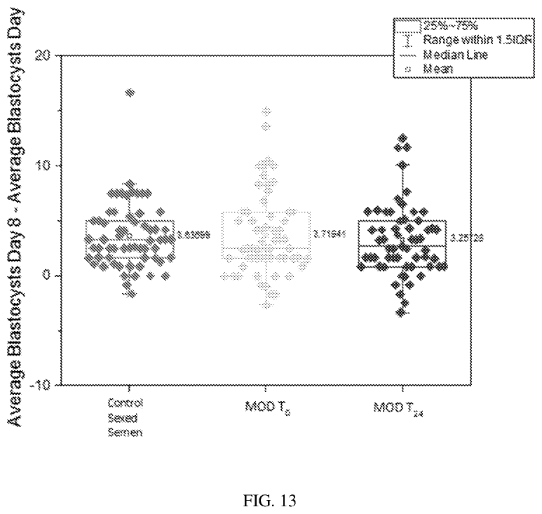

The difference in achieving significance with both SVFEM treated groups for day 7 blastocyst conversion and with only T₀ SVFEM on day 8 raised the question of whether the number of blastocysts in the treatment groups had reached the blastocyst stage earlier than the controls or if the control blastocyst numbers were increasing more rapidly than the SVFEM treated groups between day 7 and day 8. When looking at the difference between day 7 and day 8 one can see that on average, all groups increased by about 3.5%, suggesting that the increases between day 7 and day 8 are consistent among all tested groups. Further, there are no significant differences in the percent difference in blastocyst day 7 to day 8 between the groups that would immediately explain the difference in significance between day 7 and day 8, overall ANOVA of the percent change of blastocysts between day 7 and day 8 gave a p-value of 0.723. However, the ANOVA looking at the average change from day 7 to day 8 had low power, 0.10. Boxplot comparing the measured differences can be seen in FIG. 13. From the data collected the cause of the loss of significance between T₂₄ SVFEM and control sexed semen cannot be determined.

Increases in fertilization and embryonic development events during this IVF trial could be attributed to a few different theoretical mechanisms. The first is that the sperm cells treated with SVFEM became more responsive to the dose of heparin in the BO-IVF media. Previous work by Evergen Biotechnologies, Inc. demonstrated that custom calculated levels of heparin in the fertilization media, can lead to increased embryo development in IVF performed with sexed semen. This is because ejaculates from different sires are differentially sensitive to activation with heparin (Xu et al., 2009). The heparinized media used in this trial had a constant dose of heparin, so it is possible that SVFEM extender affected the spermatozoa's capacity to react to heparin, altering fertilization and embryonic developmental rates. To test this hypothesis the SVFEM treated insemination doses could be evaluated in an IVF trial that uses variable heparin concentrations to determine if monospermic fertilization and blastocyst conversion are altered with changes in heparin dose.

A second possible mechanism is that the SVFEM may be capacitating the spermatozoa during incubation with the SVFEM extender. Capacitation must be complete to allow interaction with the zona pellucida (Yanagimachi, 1994). While the full process of capacitation is not fully understood, calcium helps trigger the process of capacitation. Calcium is also known to increase hyperactivation which is activated by capacitation (Lopez and Jones, 2013). Capacitation completion leads to hyperactivation which in turn leads to more effective motion and fertilization (Smith and Yanagimachi, 1989). Knowing that SVFEM formulation contains calcium it is possible that a prolonged incubation leads to increased capacitation, which in turn leads to spermatozoa ready to fertilize after a shorter incubation period compared to their untreated control group. To test this theory, the motility parameters of frozen-thawed SVFEM treated and paired control semen would be thawed and incubated in the BO-IVF capacitation media and compare measured velocities. Capacitation can also be assayed using fluorescent microscopy to visualize the acrosome reaction.

A third possibility is that antioxidants present in the SVFEM extender could minimize oxidative stress and DNA damage in the sperm during the sexing process, ultimately improving embryo development. UV light, used to excite Hoechst in all spermatozoa during the sexing process, produces ROS which can lead to strand breaks and oxidized base pair damage (Richter et al., 1988; Kong et al., 2009). DNA damage in spermatozoa induced by radiation prevents later stages of embryo development but does not change initial fertilization or cleavage rates (Fatehi et al., 2006). Both cleavage rates and blastocyst development are significantly improved in the SVFEM treated groups compared to controls, suggesting SVFEM extension may be influencing more than a single difference in cellular behavior.

The overarching conclusion from the data collected during the IVF trial was that in all assessed parameters, T₀ SVFEM performed better in IVF than paired controls. T₂₄ SVFEM also performed better than controls in cleavage and blastocyst day 7 conversion per oocyte, and in the other measured outcomes, did not perform significantly different than the controls. In fact, the T₂₄ SVFEM treated samples when compared to T₀ SVFEM and control semen in an ANOVA showed that it was not different from either group, even when T₀ SVFEM was significantly greater than the control non-extended semen. This indicates that the T₂₄ SVFEM samples are performing at least as well as the controls, while also not performing significantly less than the T₀ SVFEM. Extending with SVFEM and incubating for up to 24 hours prior to sexing, therefore, produces a sexed semen product which fertilizes oocytes and results in early embryonic development events similar to or better than non-extended sexed semen. The increase in time before beginning the sexing process, while maintaining the ability to fertilize oocytes and produce cleaved and morphologically normal blastocysts normally is a significant improvement on current procedures, as it the increase in the number of sexed semen insemination doses per ejaculate volume collected. Though this early phase of testing did not assess the viability of the presumptive blastocysts produced, the creation of a visually assessed normal blastocyst structures does confirm that the SVFEM treated cells are capable of fertilization and completion of early embryonic development beyond the activation of the embryonic transcripts. Future testing must be done to evaluate fertilization efficiency in vivo and pregnancy and calving rates before SVFEM can be implemented in our sexed semen product.

### Example 2

Ejaculates for two sires were processed as split ejaculates, half generating control sexed semen and the other half generating 24 hour-SVFEM extended then sexed semen. The relative conception rate is reported as [(SVFEM-extended % pregnant)/(control % pregnant)]*100.

For sire 1, the relative SVFEM/control conception rate (relative conception rate) was 92%.

For sire 2, the relative conception rate was 126%.

Average between the two is 109%.

This data indicates that pregnancies can be achieved with 24-hour SVFEM-extended semen.

The initial data points, conception rates appear similar to control.

### Example 3

TABLE 2
Pearson's correlation coefficient values relating IVF outcomes
to velocity measurements taken nost-thaw in test diluent media.
Those highlighted indicate slope values significantly different from zero at
α = 0.05. Reported R² are the adjusted R² values.

| | | Day 7 | Day 8 |
|---|---|---|---|
| Velocity | Cleaved | Blastocyst | Blastocyst |
| Parameter | Percent | Percent | Percent |
| Motile Conc. P- | 0.304 | 0.539 | 0.692 |
| value | | | |
| Motile Conc. R² | 0.0005 | −0.005 | −0.006 |
| ALH P-value | 0.265 | 0.37 | 0.213 |
| ALHR² | 0.002 | −0.001 | 0.004 |
| BCF P-value | 0.0001 | 0.012 | 0.025 |
| BCF R² | 0.1 | 0.04 | 0.031 |
| LIN P-value | <0.00001 | 0.379 | 0.512 |
| LIN R² | 0.12 | −0.002 | −0.004 |
| STR P-value | <0.00001 | 0.345 | 0.461 |
| STR R² | 0.172 | −0.0007 | −0.003 |
| VAP P-value | <0.00001 | 0.018 | 0.01 |
| VAP R² | 0.173 | 0.035 | 0.042 |
| VCL P-value | 0.0003 | 0.051 | 0.032 |
| VCL R² | 0.09 | 0.021 | 0.027 |
| VSL P-value | <0.00001 | 0.015 | 0.011 |
| VSLR² | 0.246 | 0.037 | 0.041 |
| WOB P-value | 0.001 | 0.556 | 0.702 |
| WOB R² | 0.07 | −0.005 | −0.007 |

### Media Recipes:

All chemicals are purchased through Sigma-Aldrich unless otherwise specified.

Tyrode's: 1 liter in H₂O

### 94.5 mM NaCl

### 3 mM KCl

300 μM Na₂HPO₄

### 10 mM NaHCO₃

400 μM MgCl₂*6H₂O
Osmolarity 180-190 mOsm
Filter with 0.22 μm bottle top PTFE filter

Stain TALP:

1 liter in Tyrode's
25 mM Sodium lactate syrup (60% w/w)

### 5 mM Glucose

### 40 mM HEPES

### 0.5 mM Sodium Pyruvate

3 mg/mL BSA (Fraction V)
pH 7.6
Osmolarity 290-320 mOsm
Filter with 0.22 μm bottle top PTFE filter

Red TALP:

### 1928 mL Stain TALP

### 60 mL Egg Yolk

### 12 mL 1% Red Food Dye

pH 5.8
Settle for 24 hours before decanting

Motility Diluent:

### 300 mL Stain TALP

### 600 mL Red TALP

Filter with 0.22 μm bottle top PTFE filter

TRIS A:

### 1440 mL Sterile Milli-Q H₂O

### 160 mL 10×TRIS Stock

### 400 mL Egg Yolk

### Invert to mix

Wait 48 hours before decanting
Add GTLS to 2% v/v decanted TRIS A

TRIS B:

### 1012 mL Sterile Milli-Q H₂O

### 200 mL 10×TRIS Stock

### 66 mL Egg Yolk

### 720 mL Glycerol

### 2.0 mL Green Food Color

### 0.1% GTLS v/v TRIS B

Packaging Extender:

### 400 mL TRIS A

### 80 mL TRIS B

Gentamycin Sulfate Solution:

52.0 g Gentamicin Sulfate (powdered, Amresco #0304)

### 500 mLs Sterile Milli-Q H₂O

Tylosin Solution:

### 8.85 Tylosin (Tylosin Tartrate; Midwest Vet Supply)

### 500 mL Sterile Milli-Q H₂O

GTLS:

### 4 mL Gentamicin Sulfate Solution

### 3 mL Tylosin Solution

### 3 mL Linco-Spectin Stock (50 mg Lincomycin/100 mg Spectinomycin; Midwest Vet Supply)

QC Diluent:

10 mL Bovine TF Sheath fluid (Chata Biosystems Fort Worth, Tex. USA)

### 2 mL TRIS B

### 240 μLs 1% Red Food Dye

Mg²⁺ and Ca²⁺ free PBS:

### 8 g NaCl

### 0.2 g KCl

1.44 g Na₂HPO₄
0.24 g KH₂PO₄
pH 7.4, bring to 1 liter in diH₂O, filter and store at room temperature

Lysis Solution:

### 2.5 M NaCl

### 0.1 M EDTA Na₂

### 10 mM TRIS-HCl

pH 10, filter, store at 4° C.

10% Triton X-100:

### 10 mL Triton X-100

90 mL lysis buffer

### Store at 4° C.

Neutralization Buffer:

### 0.4 M TRIS

pH 7.5, store at room temperature

Electrophoresis Solution:

### 0.3 M NaOH

### 1 mM EDTA Na₂

pH 13
make fresh each use and pre-chill to 4° C. at least 1 h

Enzyme Reaction Buffer:

### 40 mM HEPES

### 0.1 M KCl

### 0.5 mM EDTA Na₂

0.2 mg/mL BSA
pH 8.0
made as 10× stock and aliquots frozen at −20° C.

Agarose:

Agarose dilutions made with diH₂O
Low melting point and standard agarose were purchased through Invitrogen

Pre-Coated Agarose Slides:

100 mL 1% agarose is melted in a glass beaker. Single sided frosted glass slides were dipped in the agarose. Backs of the slides were wiped clean. Slides were laid out at room temperature, protected from dust, overnight to dry. The next day the slides can be replaced in the slide box for storage

### Optimization of Methods for Endonuclease-Mediated Alkaline Comet Assay

Comet assays have been used to quantify single cell DNA damage in many cell types since initial assay development in 1984 by Ostling and Johanson. Two different comet techniques using different electrophoresis buffer pH conditions emerged; an alkaline comet assay which is reported to quantify the extent of double-strand breaks (Singh et al., 1988), and a neutral comet assay, with greater specificity for single-strand breaks (Olive et al., 1991). Applications for the comet assay have increased with the ability to add glycosylases and nucleases digestion steps to reveal other types of DNA damage, in addition to strand breaks (Piperakis 2008). Specific endonucleases cleave only at sights where DNA has been oxidized; the endonuclease converts the oxidized site to a strand break which is then quantifiable following standard comet assay electrophoresis (Collins et al., 2008).

DNA integrity in sperm has been assessed using endonuclease-mediated alkaline comet assays and related to fertility profiles, confirming correlations between DNA damage quantified by the comet assay and sperm fertility (Bittner et al., 2018 (bovine); Hughes et al., 1996 (human); Mukhopadhyay et al., 2010 (bovine)). Induced increases of ROS by dosing with hydrogen peroxide followed by endonuclease-mediated alkaline comet assay showed significant increases in DNA fragmentation in the sperm (Hughes et al., 1996). Evaluation of fertilizations with artificially oxidatively stressed spermatozoa has shown that fertilization rates are negatively impacted (Aitken et al., 2009). Another study in bovine showed decreased embryo development when oocytes were fertilized with spermatozoa that had been exposed to oxidative damage specifically (Bittner et al., 2018).

Sperm is particularly sensitive to exogenous DNA insults because they lack most DNA repair mechanisms (Aitken and Baker, 2006). Two processes that may increase ROS (and therefore DNA damage) during sperm sexing are UV light that is used to excite the DNA binding dye and glycerol used in the cryoprotection step (Aitken et al., 2015; Farber, 1994). Low levels of ROS are required for normal sperm activation and capacitation (Agarwal et al., 2003; Aitken and Baker, 2002), but high concentrations of intracellular ROS have been shown to increase single and double strand breaks (Aitken and Krausz, 2001), and modification of bases, deletions, and frame shifts (Twigg et al., 1998b; Duru et al., 2000). These DNA insults accumulate in sperm because the cells are deficient in antioxidant enzymes (Aitken and Fisher, 1994). Accumulated DNA damage can negatively impact fertilization and embryo development (Aitken et al., 2009 and Bittner et al., 2018).

DNA double and single-strand breaks were quantified in sexed and un-sexed sperm in the previous literature which concluded that sexed semen does not exhibit increased levels of DNA damage compared to un-sexed semen. (Boe-Hansen et al., 2005; Gonsalvez et al., 2010). However, these assessments were done on sperm sexed using the Beltsville method and not the sexing procedure disclosed in the instant application, in which UV laser ablates the unwanted cell populations. This difference could expose the cells to more UV damage and increase induced DNA damage, and sexed semen produced by UV laser ablation has yet to be assessed in this way. Additionally, previous studies on sexed semen DNA integrity did not use any endonuclease treatment to expose specific DNA base pair modification damages, which are important in understanding ROS specific damage.

SVFEM contains antioxidants and phospholipids which can act as an oxidative sink, and therefore has the potential to mitigate ROS accumulation in sperm. An endonuclease-mediated alkaline comet assay can be used to determine whether SVFEM extension mitigates oxidative DNA modification which occurs during sperm sexing. Due to the improved embryo development and fertilization rates in SVFEM-treated samples that are similar to reported literature outcomes in comparisons of oxidatively stressed sperm fertilization, it is hypothesized that SVFEM extension mitigates oxidative DNA damage caused by sexing.

Materials and Methods:

Medias used in the following experiments are outlined above.

Comet Assay: The overall comet experimental outline followed the steps described in Hartmann et al., 2003; briefly, cells were isolated, embedded in low-melting agarose gels, exposed to lysis conditions, equilibrated in electrophoresis solution, electrophoresed for a minimum of 30 minutes at 0.7 V/cm, neutralized, stained, and imaged. Lysis solution detergent mixtures and concentrations varied, as did the duration and temperature of the lysis conditions. For all experiments, a positive control slide, treated with 200 μM H₂O₂ was included for which one would expect to see induced DNA damage as described previously in Hughes et al., 1996, and a negative control slide treated with diH₂O for which one would expect only baseline DNA damage. Experiments included endonuclease treatment (Endo III) as well, for which one would expect to see increased DNA damage due to induced strand breaks (Kushwaha et al., 2011). This led to the inclusion of 4 treatment groups for the comet assay: H₂O₂+Endo III (positive/positive slide), H₂O₂+1× enzyme reaction buffer (positive/negative slide), diH₂O+Endo III (negative/positive), and diH₂O+1× enzyme reaction buffer (negative/negative slide).

Alterations to the lysis buffer included additions of sodium dodecyl sulfate (SDS), or N-lauryl-sarcosine to differ the chemical composition of the surfactant. Both have been used to successfully liberate the DNA from the matrix in comet assays (Ward, 2013; Bittner et al., 2018). Changes in the temperature of lysis and the duration were altered for each experiment based on the chemical composition of the lysis buffer and the outcomes of previous comet lysis attempts in the lab. The inclusion of proteinase K for protamine removal and dithiothreitol (DTD) for breakage of disulfide bonds was also tested based on previously published sperm comet data (Donnelly et al., 2000; Hughes et al., 1996).

All experiments began with an 80% BoviPure™ gradient to remove dead or sliced spermatozoa from the sperm preparation, so DNA damage is being measured in only spermatozoa that survived sexing and the freezing process. A live/dead stain, ethidium monoazide bromide (EMA) which covalently binds the DNA and whose excitation/emission wavelengths do not overlap Hoechst 33342 used to visualize the comet tails, was included before this gradient and centrifugation steps to allow the exclusion of cells that were dead before the comet assay, but not removed during the BoviPure™ step, from being quantified during image analysis. Centrifugation was performed in a swinging bucket centrifuge at 500 g for 15 minutes, samples were aspirated to 100 μL, and resuspended in 1 mL of room temperature 1×TRIS buffer. Samples were then centrifuged a second time at 300 g for 5 minutes. The supernatant was aspirated and discarded again to the 100 μL line. Cell density was determined with either a hemocytometer, a Nucleocounter SP-100, or CASA. Sperm was diluted in 1×TRIS buffer. Hydrogen peroxide treatment for 15 minutes at room temperature was performed in Eppendorf tubes prior to addition to the slides. Sperm was mixed with 1% LMP agarose to achieve the final cell concentration in a 1:1 dilution. The sperm/LMP agarose mix was rapidly added to pre-coated agarose slides and allowed to solidify on ice. Slides were added to Coplin jars, and one of the lysis conditions was carried out. Electrophoresis equilibration with pre-chilled (4° C.) electrophoresis solution occurred at room temperature for 20 minutes before electrophoresis for 30 minutes at 0.7 V/cm 300 mA. Slides were neutralized in neutralization buffer at room temperature, and then air dried before staining and imaging.

Results: Sperm utilize a specialized DNA packaging system compared to somatic cells, replacing histones with protamines. The comet assay requires not only lysing cells to expose the nuclei in agarose gels but decondensing the DNA so it can migrate with an electrophoretic field. The first step to applying the comet assay to sexed semen was to identify lysis conditions which effectively decondense the DNA. Experimental lysis conditions included alterations of lysis temperature and duration, as well as treatment with SDS, sarcosine, proteinase K, and DTT. Concentrations varied for each active lysis component, increasing the concentrations and/or increasing incubation times in accordance with protocols previously reported.

Initial experiments used both SDS and TX-100 as the detergent components to the lysis buffer (Ward, 2013). At a 0.5% concentration in the lysis buffer, however, SDS precipitated out of solution during the 4° C. lysis. Changing the lysis temperature to room temperature avoided SDS precipitation, but nuclei remained visibly intact. Increasing the SDS concentration to 1% in the room temperature lysis step (Enciso et al., 2009) created gel integrity issues, and didn't improve DNA decondensation as demonstrated by sperm visibly intact nuclei with clear membranes in bright field microscopy, so sarcosine was tested as a substitute for SDS.

N-lauryl-sarcosine is another commonly used anionic detergent in comet assays for membrane lysis and histone and protamine removal, but remains soluble at 4° C., unlike SDS. Lysis buffer containing 0.5% or 1% sarcosine (still including 1% TX-100) was tested with a maximum lysis time of 24 hours at 4° C. (Fairbairn and O'Neill 1996; Bittner et al., 2018). Nuclear membranes were still visible after lysis, however, indicating further optimization was required.

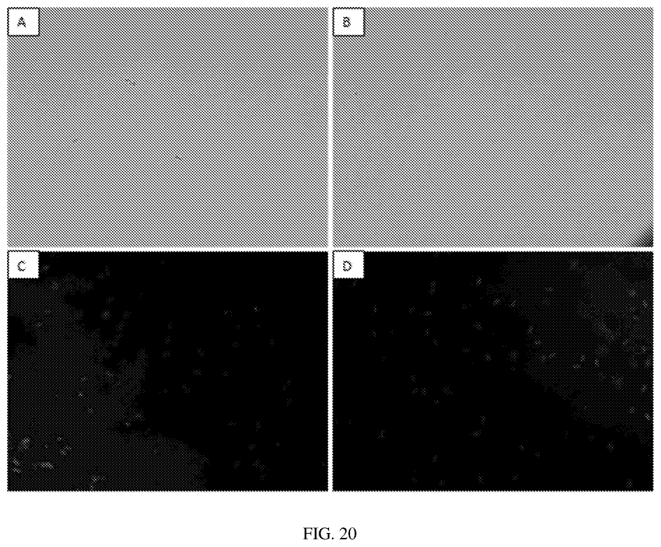

Proteinase K (1 mg/mL) is also typically used in sperm comet assay lysis buffer to remove proteins from the DNA matrix (Hughes et al., 1996). The enzyme requires a 37° C. incubation to be functionally active. DTT, a reducing agent which breaks disulfide bonds, is also typically included (2 mM-5 mM) in conjunction with Proteinase K in sperm comet assays (Castro et al., 2018; Hisano et al 2013). A Proteinase K/DTT digestion step was added after the initial detergent lysis, with incubations ranging from 3 hours to 24 hours. The added digestion step did not, however, change the observance of clear nuclear membranes on the comet assay slides (FIG. 20).

To improve detergent access to the nuclear membrane, a mechanical cell lysis step using a 1 mL Dounce homogenizer (Fisher Scientific Pittsburgh, Pa. USA) with both a small and large clearance pestle (0.06 mm and 0.13 mm clearance) prior to chemical lysis was added. Lysis conditions after mechanical lysis included a commercially available comet assay lysis buffer (Trevigen)+5 mM DTT overnight at 4° C. with proteinase K. This combination appeared to remove the nuclear membrane, which was no longer visible in bright field images, but the DNA still did not migrate after electrophoresis (FIG. 20). This suggests that the DNA is not being liberated from the protamines, or it could suggest that there is no detectable DNA damage in the raw ejaculate induced by the hydrogen peroxide positive control tested under these tested electrophoretic conditions. The lack of comet tails in the positive control suggests the assay conditions are not yet optimized.

Proposed slide analysis protocol: Images of the slides will be captured with a Zeiss Calibri while taking care to avoid comets bordering the edges of the slide and any air bubbles as these can cause distortions to the comet tails (Collins, 2004). Hoechst 33342 will be used to visualize the comet tails using 355/497 nm excitation/emission, and EMA positive cells used to exclude non-viable cells are visualized with excitation and emission of 504/600 nm. EMA covalently bonds with DNA molecules and therefore cannot fluoresce in cells that had intact membranes during staining, and this bond would not be changed during cell lysis and DNA denaturation steps. At least 50 cells per duplicate slide will be quantified to provide statistical rigor (Collins, 2004, Hartmann et al., 2003, Boe-Hansen et al., 2005, Hughes et al., 1996),

Using the OpenComet plugin for ImageJ, the ‘Olive Moment,’ which quantifies a multiplicative value of the tail length and fluorescent intensity, will be calculated and analyzed (Tice et al., 2000; Olive and Bananth, 1993). The Olive Tail Moment will be used to compare the levels of DNA damage measured among the treatment groups.

Proposed statistical analysis: Statistical analysis will be performed using OriginPro 2018 64-bit software. Based on the data expected to be collected one-way ANOVAs will be used to compare among SVFEM and control treatment groups.

Discussion: Optimization of the endonuclease-mediated alkaline comet assay is ongoing. Images from the attempt to generate comet tails showed compact DNA in a sperm head shape, suggesting that the DNA helix from the nuclear matrix was not fully liberated. Due to the increased compaction of the DNA in spermatozoa compared to somatic cells comet assays for sperm need to be specifically adapted. Specific alterations include SDS addition for decondensing sperm chromatin and additions of DTT for that same purpose (Rousseaux and Chevret, 1995). An additional alteration commonly used is the inclusion of proteinase K to break down protamines, which sperm use for DNA packaging instead of histones (Singh et al, 1989). Optimizing detergent composition of the lysis solution continues and may include replacing Triton X-100 with NP-40 which is a similar nonionic detergent and inclusion of lithium diiodosalicyclate (LIS) for DNA decondensation.

DNA damage decreased: Should comet data indicate that DNA modification in SVFEM treated groups is less than that in controls, it will suggest that the SVFEM extension protects sperm from DNA modification during the sexing process. Therefore, it is hypothesized that this decrease in damage is due to a decrease in ROS given the antioxidants present in the SVFEM extender, design experiments to assay ROS accumulation in sperm+/−SVFEM extension at various stages of the sexing process would be prepared. Insemination units would be generated from sperm extended with SVFEM with and without antioxidants (SVFEM-A). Comet analysis using Endo III would be used to assess DNA damage present in these tested groups. Should the SVFEM-A group present with DNA damage similar to the unextended controls it could be infered that the antioxidant supplementation is specifically mediating DNA damage.

ROS content of sperm could be measured using a spectrophotometer (Balamurugan et al., 2018). This experimental approach relies on the color change in transition metals as they are exposed to increasing levels of ROS in solution (Hyashi et al., 2007). This approach would also allow tracking where increases of detrimental levels of ROS accumulate and determine whether SVFEM prevents that accumulation.

No differences in DNA damage: Should DNA damage assessed for all three treatment groups present with no significant differences, it would be hypothesized that the differences in IVF outcomes would be due to either differences in motility profiles, or capacitation state.

Motility metrics in media without activation factors have been collected during the trial. Previous studies looking at CASA velocity metrics without dilution media have found relationships with IVF outcomes (Kasimanickam et al., 2006). Correlations between measured motility parameters and IVF outcomes can be calculated to determine if any relationships exist. Additional velocity measurements in heparinized media, used during IVF fertilization procedures, will be collected. Heparin is an activator of the acrosome reaction and is needed to activate sperm in vitro for fertilization (Parrish et al., 1988). Heparin changes sperm velocity characteristics (Chamberland et al., 2001), and future studies may determine whether velocity measurements in heparinized media correlate with the IVF outcomes.

Differences incapacitation state between SVFEM treated and unextended control semen could also affect their behavior in IVF. The SVFEM extender could be accelerating the capacitation reaction specifically through calcium signaling (Lopez and Jones, 2013), or through another pathway. The capacitation reaction is required to be complete before fertilization can occur, and heparin activation of this reaction takes a minimum of four hours (Parrish, 2013). However, if the capacitation state progresses in the SVFEM treated ejaculates ahead of that in the unextended controls, the sperm cells would be competent to fertilize more rapidly than the unextended controls. Capacitation state can be assessed through fluorescent staining of the acrosome because capacitation culminates in the completion of the acrosome reaction (Roldan and Harrison, 1990).

No detectable DNA damage in sexed semen samples: Should the comet assay identify quantifiable DNA damage in positive control cells, those samples treated with H₂O₂ to induce DNA damage, but no quantifiable damage be evident in the sexed semen samples that could confirm the use of this endonuclease mediated alkaline comet assay to determine oxidative DNA damage in bovine sexed semen. It would also indicate that DNA damage does not play a role in the differences quantified in the IVF trial. Alternately it could indicate that the induced DNA damage presented in the sexed semen is not visible using the endonuclease Endo III which only cleaves oxidized pyrimidines. Altering the endonuclease treatment to formamidopyrimidine-glycosylase or hOGG1, which cleave at different oxidized base pairs, might also be required to fully determine the exact type of DNA damage present.

### Example 4

The inventive media is formulated as follows (designated SVFEMSVFEM): Basic salt media CEP2 (Table 3) was combined with the additives in Table 4.

TABLE 3
Basic salt media (CEP2)

| Chemical | 1X Concentration | |
|---|---|---|
| NaCl | 15 | mM |
| KCl | 7 | mM |
| CaCl₂(H₂O)₂ | 3 | mM |
| MgCl₂(H₂O)₆ | 4 | mM |
| NaHCO₃ | 11.9 | mM |
| NaH₂ PO₄ dihydrate | 8 | mM |
| KH₂PO₄ | 20 | mM |
| Fructose | 55 | mM |
| Sorbitol | 1 | g/L |
| Bovine Serum Albumin | 2 | g/L |
| TRIS base | 133.7 | mM |
| Citric acid | 42.9 | mM |

TABLE 4
Additive concentrations for SVFEM

| Component | Concentration | |
|---|---|---|
| phosphatidylserine | 5 mM (2 mg/mL) | |
| decursin | 1 | uM |
| zinc chloride | 10 | ug/mL |
| coenzyme Q10 | 50 | ug/mL |
| aspirin | 1 | mM |
| linolenic acid | 5 | ng/mL |
| fatty acid supplement | 1 | uL/mL |
| D-aspartic acid | 500 | ug/mL |
| NaF | 6 | mM |

In the basic salt media, TRIS base may be substituted with 20 mM HEPES ((4-(2-hydroxyethyl)-1-piperazineethanesulfonic acid), an organic chemical buffering agent. The fatty acid supplement is a commercially available mixture from Sigma Aldrich.

Sperm samples (n=3) stored in media SVFEM showed no measurable reduction in motility after 24 hours of holding. A control sample using conventional media showed a significant reduction in motility over the same time period. See FIG. 31.

Control/conventional media used are those that are known in the art, and examples include but are not limited to, salt or sugar (saccharide) solutions, including glucose solutions. Control media may also include standard buffer solutions known in the art.

NaF would not be added to the media if applied to conventional semen processing. Thus, some alternative formulations do not contain NaF, which can inhibit motility. However, testing demonstrated improved efficacy for the number of motile cells which survive a freeze/thaw after 24 hours' extension. Sample processing diluted the semen sufficiently to reverse the motility inhibition provided by NaF. Thus, NaF is included in SVFEM and there is also potential to use other motility inhibitors identified in the literature in combination with the media. See FIG. 32 where the effect of NaF is shown. The plot in FIG. 2 illustrates numbers of post-thaw motile cells per sample in ejaculates processed same day or after a 24-hour hold, with varying concentrations of NaF. The concentration is denoted along the x-axis and has a range from 0-6 mM NaF.

### Example 5

Semen sample creation: The media of Example 1 is mixed with semen in a 1:1 volume ratio. Optimal performance is seen when the media was warmed to 37° C. prior to mixing with Angus bull semen, and when the media is added to semen between 10 and 30 minutes post-ejaculation. The media remains effective, however, when mixed with cells 1 hour after ejaculation. After the addition of the media, cells are stored at 19° C. FIG. 3 shows that the inventive media SVFEM shows a 10% increase in motile cells over conventionally processed samples. In FIG. 33 p=0.017, in a paired t-test.

### Example 6

Samples were tested for maintenance of motility of cells after 24 hours of storage. Semen samples from Angus bulls (n=4) were taken and tested at the collection, then after storing in the media of Example 1 (SVFEM) for 24 hours. The samples retained adequate motility after storage. See FIG. 4.

Additionally, the same sample was stained for a sexing process (which involves the binding of an excitable dye to the DNA). Motility of cells in SVFEM was also maintained after this step. See also FIG. 34.

### Example 7

Another test was performed to compare samples after 24-hour storage. Semen samples from Angus bulls (n=10) were taken and combined with media. Samples using the inventive media SVFEM were compared to control samples using conventional media. Samples were analyzed to determine whether motility of cells was lost due to storage. The use of the inventive media (SVFEM) allows for maintenance of motility of cells after 24-hour storage. See FIG. 5.

Additionally, the same samples were stained for a sexing process (which involves the binding of an excitable dye to the DNA). Motility of cells was also maintained after this step when using the inventive media (SVFEM). See also FIG. 35.

### Example 8

The SVFEM media was tested for the creation of samples for further use (e.g., AVF or AI). These samples, or straws, contain sperm cells that have been processed, in this instance, cells have been sexed. Straws were tested for motile cells per sample. Samples using the inventive media SVFEM (n=8) stored for 24 hours, showed similar motility of cells to those using a control media (n=3). See FIG. 36. Samples were packaged at 2.3×106 cells/straw.

### Example 9

The use of the inventive media SVFEM can improve sexed semen fertilization rates in IVF and/or AI. Samples using SVFEM were tested for production of blastocysts/fertilized oocytes. Sexed samples (n=10) using both SVFEM and a control media were compared to conventional samples (not sexed). SVFEM shows a similar number of blastocysts per oocytes fertilized between the two-sexed samples. See FIG. 37. When further comparing samples (n=3 ejaculates/treatment, paired) of sexed semen, using a conventional media versus SVFEM, an increase in blastocysts per oocytes fertilized is seen when the inventive media is used. See FIG. 38.

### Example 10

The effect of eliminating ingredients from SVFEM media was investigated. When comparing motility for samples stored for 24 hours, dropping out the additives on an individual basis does not eliminate efficacy for the SVFEM hold media, except in the case of phosphatidylserine (PS). See FIG. 39. PS is a key ingredient and removal of PS causes a drop in motility.

### Example 11

Media may be formulated in a three-part mixture for ease of use. The three parts are as follows: (1) CEP2 supplemented with ZnCl, Fatty Acids, D-aspartate, stored at 4° C.; (2) 1000× organic stock solution containing decursin, aspirin, coenzyme Q10, linolenic acid, stored at −20°; (3) Phosphatidylserine, stored at −20° C. The three parts are then mixed in the appropriate ratios at the time of use. Semen samples prepared from stock SVFEM media prepared in this way show maintenance of motility after 24 hours similar to SVFEM made by combining all ingredients. See FIG. 40.

All publications and patents mentioned herein are hereby incorporated by reference in their entirety as if each individual publication or patent was specifically and individually indicated to be incorporated by reference.

While specific aspects of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### BIBLIOGRAPHY

Agarwal, A., Saleh, R. A., Bedaiwym, A. (2003). “Role of reactive oxygen species in the pathophysiology of human reproduction.” Fertility and Sterility 79:829-843.
Aitken, R., Baker, M., Nixon, B. (2015). “Are sperm capacitation and apoptosis the opposite ends of the continuum driven by oxidative stress?” Asian J Androl 17: 633-639.
Aitken, R. J., Baker, M. (2006). “Oxidative stress, sperm survival and fertility control.” Mol Cell Endocrinol 250:66-69.
Aitken, R. J., Baker, M. A. (2002). “Reactive oxygen species generation by human spermatozoa: a continuing enigma.” Int J of Andro 25:191-194.
Aitken, R. J., De Iuliis, G. N., et al (2009). “Biological and clinical significance of DNA damage in the male germ line.” Int J Androl 32:46-56.
Aitken, R. J., Fisher, H. (1994). “Reactive oxygen species generation and human spermatozoa: the balance of benefit and risk.” Bio Essays 16:259-267.
Aitken, R. J., Krausz, C. (2001). “Oxidative stress, DNA damage and the Y chromosome.” Reproduction 122: 497-506
Almquist, J. O. (1973). “Effects of sexual preparation on sperm output, semen characteristics and sexual activity of beef bulls with a comparison to dairy bulls.” J Anim Sci 36:331-336.
Almquist, J. O., Glantz, P. J., Shaffer, H. E. (1949) “The effect of a combination of penicillin and streptomycin upon the livability and bacterial content of bovine semen.” J Dairy Sci 32: 183-190.
Alvarez, J. G., Storey, B. T., (1992). “Evidence for increased lipid peroxidative damage and loss of superoxide dismutase activity as a mode of sublethal cryodamage to human sperm during cryopreservation.” J Androl 13 (3): 232-241.
Bacinoglu, S., Tas, M. et al. (2007). “The potential fertility estimation capacity of the hypoosmotic swelling test, the thermal stress test and a modified cervical mucus penetration test in the bovine.” Anim Reprod Sci doi:10.1016/j.anireprosci.2007.01.014.
Balamurugan, B., Ghosh, S. K., et al. (2018). “Parital deoxygenation of extender improves sperm quality, reduces lipid peroxidation and reactive oxygen species during cryopreservation of buffalo (Bubalus bubalis) semen.” Ani Repro Sci 189:60-68.
Barcelo-Fimbres, M., Campos-Chillon, L. F., Seidel Jr, G. E. (2011). “In vitro fertilization using non-sexed and sexed bovine sperm: sperm concentration, sorter pressure, and bull effects.” Reprod Dom Anim 46:495-502.
Barker, D. J. “The fetal and infant origins of adult disease.” BMJ 301:1111
Bartlet, F. D., VanDemark, N. L. (1962). “Effect of diluent composition on survival and fertility of bovine spermatozoa stored in carbonated diluents.” J Dairy Sci 45: 360-367.
Bermejo-Alvarez, P., Lonergan, P, Rath, D., et al. (2010). “Developmental kinetics and gene expression in male and female bovine embryos produced in vitro with sex-sorted spermatozoa.” Repro, Fert, Devel 22:426-436.
Bittner, L., Wyck, S., Herrera, C. et al. (2018). “Negative effects of oxidative stress in bovine spermatozoa on in vitro development and DNA integrity of embryos.” Repro, Fert, and Devel doi.org/10/1071/RD17533
Blom, E. (1950). “Interpretation of spermatic cytology in bulls.” Fertil Steril 1: 223-238
Blondin, P., Beaulieu, M., Fournier, V., et al. (2009). “Analysis of bovine sexed sperm for IVF from sorting to the embryo.” Theriogenology 71:30-38.
Boe-Hansen, G. B., Morris, I. D., et al. (2005). “DNA integrity in sexed bull sperm assessed by neutral comet assay and sperm chromatin structure assay.” Theriogenology 63:1789-1802.
Bratton, R. W., Foote, R. H., et al. (1956). “The relative usefulness of combinations of laboratory tests for predicting the fertility of bovine semen.” J Dairy Sci 39: 1542-1549.
Cassou, R. (1964). “La methode des pailletes en plastique adaptee a la generalization de la congelation.” 5^{th} Int Cong Anim Reprod Trento, Italy. 4:540-546.
Cebrian-Serrano, A., Salvador, E. G. R., et al. (2012). “Effect of the bovine oviductal fluid on in vitro fertilization, development and gene expression of in vitro-produced bovine blastocysts.” Repro in Dom Anim 48(12):331-338.
Chamberland, A., Fournier, V., et al. (2001). “The effect of heparin on motility parameters and protein phosphorylation during bovine sperm capacitation.” Theriogenology 55:823-835.
Collins, A. R., Duthie, S. J., Dobson, V. L. (1993). “Direct enzymic detection of endogenous oxidative base damage in human lymphocyte DNA.” Carcinogenesis 14:1733-1735
Collins, A. R. (2004). “The comet assay for DNA damage and repair.” Molecular Biotechnology 26:249.
Davis, I. S., Bratton, R. W., Foote, R. H. (1963). “Livability of bovine spermatozoa at 5, −25, and −85° C. in TRIS-buffered and citrate buffered yolk-glycerol extenders.” J Dairy Sci 46: 333-336.
de Boo., H. A., Harding, J. E., “The developmental origins of adult disease (Barker) hypothesis.” Anzjog 46(1):4-14.
Donnelly, E. T., O'Connell, M., et al. (2000). “Differences in nuclear DNA fragmentation and mitochondrial integrity of semen and prepared human spermatozoa.” Human Reproduction 15(7):1552-1561.
dos Santos Hamilton, T. R., de Castro, L. S., (2016). “Induced lipid peroxidation in ram sperm: semen profile, DNA fragmentation and antioxidant status.” Reproduction 151: 379-390
Duru, N. K., Morshedi, M., Oehninger, S. (2000). “Effects of hydrogen peroxide on DNA and plasma membrane integrity of human spermatozoa.” Fertil Steril 74:1200-1207.
Elferink, M., Schierhorn, F. (2016). “Global demand for food is rising. Can we meet it?” Harvard Business Review https://hbr.org/2016/04/global-demand-for-food-is-rising-can-we-meet-it
Enciso, M., Sarasa, J., et al. (2009). “A two-tailed comet assay for assessing DNA damage in spermatozoa.” Repro BioMedicine Online B18B(5):609-616.
Fairbairn, D. W., O'Neill, K. L. (1996). “The neutral comet assay is sufficient to identify an apoptotic ‘window’ by visual inspection.” Anoptosis 1:91-94.
Farber, J. L. (1994) “Mechanisms of cell injury by activated oxygen species.” Environ Health Perspect 102(Suppl 10): 17-24.
Fatehi, A. N., Bevers, M. M., et al. (2006). “DNA damage in bovine sperm does not block fertilization and early embryonic development but induces apoptosis after the first cleavages.” Journal of Andrology 27(2): 176-188.
Foote, R. H. (2002). “The history of artificial insemination: Selected notes and notables.” J Anim Sci 80: 1-10.
Foote, R. H., Bratton, R. W. (1949). “The fertility of bovine semen cooled with and without the addition of citrate-sulfanilamide-yolk extender.” J Dairy Sci 32: 856-861.
Foote, R. H., Bratton, R. W. (1950). “The fertility of bovine semen in extenders containing sulfanilamide, penicillin, streptomycin, and polymyxin.” J Dairy Sci 33: 544-547.
Foote, R. H., Bratton, R. W., (1963). “Effect of sperm losses in semen collection equipment on estimated sperm output by bulls.” J Dairy Sci 46:242-244.
Foote, R. H., Gray, L. C., et al. (1960) “Fertility of bull semen stored up to four days at 5° C. in 20% egg yolk extenders.” J Dairy Sci 43: 1330-1334.
Gardner, D. K., Sakkas, D. “Assessment of Embryo Viability: The Ability to Select a Single Embryo for Transfer—a Review.” Placenta 24:S5-S12.
Garner, D. L. (2006). “Flow cytometric sexing of mammalian sperm.” Theriogenology 65(5): 943-957.
Garner, D. L., Seidel Jr., G. E. (2003). “Past, present and future perspectives on sexing sperm.” Can J of Anim Sci 83(3): 375-384.
Gliozzi, T. M., Turri, F., et al. (2017). “The combination of kinetic and flow cytometric semen parameters as a tool to predict fertility in cryopreserved bull semen.” Animal 11(11): 1975-1982.
Gonsalvez, J., Ramirez, M. A., et al (2010). “Sex-sorted bovine spermatozoa and DNA damage: I. Static features.” Theriogenology 75:197-205.
Gonsalvez, J., Ramirez, M. A., et al (2010). “Sex-sorted bovine spermatozoa and DNA damage: II. Dynamic features.” Theriogenology 75:206-211.
Greve, T., Madison, V. (1991). “In vitro fertilization in cattle: A review.” Repro Nutri Devel 31(2):147-157.
Greve, T., Madison, V., et al. (1993). “In vitro production of bovine embryos: A progress report and the consequences on the genetic upgrading of cattle populations.” Anim Repro Sci 33(1):51-69.
Hafs, H. D., Hoyt, R. S., Bratton, R. W. (1959). “Libido, sperm characteristics, sperm output and fertility of mature dairy bulls ejaculated daily or weekly for thirty-two weeks.” J Dairy Sci 42: 626-636.
Hartmann, A., Agurell, E., et al. (2003). “Recommendations for conducting the in vivo alkaline Comet assay.” Mutagenesis 18(1) 45-51.
Hayashi, I., Morishita, Y., et al. (2007). “High-throughput spectrophotometric assay of reactive oxygen species in serum.” Mutation Research 631(1):55-61.
Hisano, M., Erkek, S., et al. (2013). “Genome-wide chromatin analysis in mature mouse and human spermatozoa.” Nature Protocols 8(12):2449-2470.
Hohenboken, W. D. (1999). “Applications of sexed semen in cattle production.” Theriogenology 52:1421-1433.
Holden, S. A., Fernandez-Fuertes, B., Murphy, C., et al. (2016). “Relationship between in vitro sperm functional assessments, seminal plasma composition, and field fertility after AI with either non-sorted or sex-sorted bull semen.” Theriogenology doi:10.1016/j.theriogenology.2016.08.02
Huang, W., Khatib, H. (2010). “Comparison of transcriptomic landscapes of bovine embryos using RNA-Seq.” BMC Genetics 11:711
Hughes, C. M., Lewis, S. E. M., et al. (1996). “A comparison of baseline and induced DNA damage in human spermatozoa from fertile and infertile men, using a modified comet assay.” Mol Human Repro 2(8):613-619.
Ivanoff, E. I. (1922). “On the use of artificial insemination of zootechnical purposes in Russia.” J Agric Sci 12: 244-256.
Ivanov, E. I. (1907). “De la fecundation artificielle chez les mammiferes.” Arch Sci Biol 12: 377-511.
Jaloszynski, P., Kujawski, M., et al. (1999). “Genotoxicity of inhalation anesthetics halothane and isoflurane in human lymphocytes studied in vitro using the comet assay.” Mutation Research 439:199-206.
Januskauskas, A., Johannisson, A., Rodriguez-Martinez, H. (2003). “Subtle membrane changes in cryopreserved bull semen in relation with sperm viability, chromatin structure, and field fertility.” Theriogenology 60: 743-758.
Johnson, L. A., Flook, J. P., Hawk, H. W. (1989). “Sex preselection in rabbits: Live births from X and Y sperm separated by DNA and cell sorting.” Biol of Repro 41: 199-203.
Johnson, L. A., Welch, G. R. (1999). “Sex preselection: High-speed flow cytometric sorting of X and Y sperm for maximum efficiency.” Theriogenology 52: 1323-1341.
Johnson, L. A., Welch, G. R., Rens, W. (1999). “The Beltsville sperm sexing technology: High-speed sperm sorting gives improved sperm output for In Vitro fertilization and AI.” J Anim Sci 82(Suppl 2):213-220.
Kang, J. H., Hakimov, H., et al. (2008). “The negative effects of exogenous DNA binding on porcine spermatozoa are caused by removal of seminal fluid.” Theriogenology 70(8):1288-1296.
Kasimanickam, R., Nebel, R. L. et al. (2006). “Breed differences in competitive indices of Holstein and Jersey bulls and their association with sperm DNA fragmentation index and plasma membrane integrity.” Theriogenology 66:1307-1315.
Keel, B. A., Schalue, T. K. (2009). “Correlation of the bovine cervical mucus penetration test with human sperm characteristics in 1406 ejaculates.” Archives of Andrology 4:109-115.
Kong, X., Mohanty, S. K., et al. “Comparative analysis of different laser systems to study cellular responses to DNA damage in mammalian cells.” Nucleic Acids Research 37(9):e68.
Kropp, J., Carrillo, J. A., et al. (2017). “Male fertility status is associated with DNA methylation signatures in sperm and transcriptomic profiles of bovine preimplantation embryos.” BMC Genomics 18: doi:10.1186/s12864-017-3673-y
Kushwaha, S., Vikram, A., et al. (2011). “Alkaline, Endo III, and FPG modified comet assay as biomarkers for the detection of oxidative DNA damage in rats with experimentally induced diabetes.” Mutation Research 726:242-250.
Lewis, S. E. M., Boyle, P. M., et al (1995). “Total antioxidant capacity of seminal plasma is different in fertile and infertile men.” Fertil Steril 64:866-870.
Linfor, J. J, Meyers, S. A. (2002). “Detection of DNA damage in response to cooling injury in equine spermatozoa using single-cell gel electrophoresis.” J of Andrology 23(1):107-113.
Llana-Ruiz-Cabello, M., Maisanaba, S., et al. (2014), “Evaluation of the mutagenicity and genotoxic potential of carvacrol and thymol using the Ames salmonella test and alkaline, Endo III- and FPG-modified comet assay with the human cell line Caco-2.” Food and Chem Tox 72:122-128.
Lopez, K. H., Jones, R. (2013). “Human Reproductive Biology.” 4^{th} ed Academic Press, Massachusetts.
Lu, K. H., Cran, D. G., Seidel, G. E. (1999). “In vitro fertilization with flow-cytometrically-sorted bovine sperm.” Theriogenology 52: 1393-1405.
Mukhopadhyay, C. S., Gupta, A. K., et al. (2011). “Effect of cryopreservation on sperm chromatic integrity and fertilizing potential in bovine semen.” Livestock Science 136:114-121.
NEB (2018). “Comet assay-modified for detection of oxidized bases using the repair endonucleases Fpg, hOGG1, and Endonuclease III(Nth).” neb.com/protocols/1/01/01/comet-assay-modified-for-detection-of-oxidized-bases-using-the-repair-endonucleases-fpg-hoggi-and-endonuclease-iii-nth
Olive, P. L., Bananth, J. P. (1993). “Induction and rejoinging of radiation-iinduced DNA single-strand breaks: “tail moment” as a function of position in the cell cycle.” Mutation Research 294(3): 275-283.
Olive P. L, Wlodek, D., Bananth, J. P. (1991). “DNA double-strand breaks measured in individual cells subjected to gel electrophoresis.” Cancer Res 51:4671-4676.
Oliveira, L. Z., de Arruda, R. P., et al. (2012). “Assessment of field fertility and several in vitro sperm characteristics following the use of different Angus sires in a timed-AI program with suckled Nelore cows.” Livestock Science 146: 38-46.
Ostling, O., Johanson, K. J. (1984). “Microelectrophoretic study of radiation-induced DNA damages in individual mammalian cells.” Biochem Biophys Res Commun 123:291-298
Park, K. S., Kim, J. H., et al (2001). “Effects of insulin and antioxidant on plasma 8-hydroxyguanine and tissue 8-hydroxydeoxyguanosine in streptozotocin-induced diabetic rats.” Diabetes 50: 2837-2841:1171-1180.
Parrish, J. J., Susko-Parrish, J, Winer, et al. (1988). “Capacitation of bovine sperm by heparin.” Biol Reprod 38
Parrish, J. J. (2014). “Bovine in vitro fertilization: In vitro oocyte maturation and sperm capacitation with heparin.” Theriogenology 81:67-73.
Pickett, B. W., Berndtson, W. E. (1974). “Preservation of bovine spermatozoa by freezing in straws: A review.” J Dairy Sci 57: 1287-1301.
Pickett, B. W., Martig, R. C., Cowan, W. A (1961). “Preservation of bovine spermatozoa at −79 and −196° C.” Journal of Dairy Science 44(11):2089-2096.
Piperakis, S. M. (2008). “Comet assay: A brief history.” Cell Biol Toxicol 25:1-3
Polge, C. (1953). “The storage of bull semen at low temperatures.” Vet Record 65: 557-559.
Research Series 574: Arkansas Animal Science Department Report 2009; Zelpha B. Johnson and D. Wayne Kellogg, editors. Effect of a Low-Sodium, Choline-Based Diluent on Viability of Bovine Sperm Stored at Refrigerator Temperatures; P. A. Delgado, T. D. Lester and R. W. Rorie.
Richter, C., Park, J., Ames, B. (1988). “Normal oxidative damage to mitochondrial and nuclear DNA is extensive.” Proc Natl Acad Sci USA 85: 6465-6467.
Rikmenspoel, R. G., van Herpen, Eijkhout, P. (1960). “Cinematographic observations of the movements of bull sperm cells.” Phys Med Biol 5:167-182.
Roldan, E. R. S., Harrison, R. A. P. (1990). “Molecular mechanisms leading to exocytosis during the sperm acrosome reaction.” Fertilization in mammals 179-196, Norwell, Serono Symposia.
Rothschild, L. (1953). “A new method of measuring the activity of spermatozoa.” J Exp Biol 30: 178-199.
Rousseaux, S., Chevret, E. (1995). “In-vitro decondensation of human spermatozoa for fluorescence in-situ hybridization.” Mol Hum Reprod 10:2209-2213
Salisbury, G. W., VanDemark, N. L, Lodge, J. R. (1978). “Physiology of reproduction and artificial insemination of cattle. 2^{nd} ed. W.H. Freeman Co., San Francisco.
Salisbury, G. W., VanDemark, N. L. (1945). “Stimulation of livability and glycolysis by additions of glucose to the egg yolk-citrate diluent for ejaculated bovine semen.” Am J Physiol 143: 692-697.
Salisbury, G. W., Willet, E. L. (1940). “An artificial vagina for controlled temperature studies of bull semen.” Cornell Vet 30: 25-29.
Sarkaria, J. N., Bush, C., et al. (1998). “Comparison between pulsed-field gel electrophoresis and the comet assay as predictive assays for radiosensitivity in fibroblasts.” Radiation Research 150:17-22.
Schoff, P. K., Lardy, H. A. (1987). “Effects of fluoride and caffeine on the metabolism and motility of ejaculated bovine spermatozoa.” Biol Reprod 37(4):1037-1046.
Seidel Jr G E, Schenk J L, Herickhoff L A, et al. (1999). “Insemination of heifers with sexed sperm.” Theriogenology 52:1407-20.
Shannon, P. B., Curson, Rhodes, A. P. (1984). “Relationship between total spermatozoa per insemination and fertility of bovine semen stored in Caprogen at ambient temperature.” N.Z. J Agric Res 27: 35-41.
Singh, N. P., McCoy, M. T., et al. (1988). “A simple technique for quantitation of low levels of DNA damage in individual cells.” Exp Cell Res 175:184-191
Singh, N. P., Danner, D. B., Tice, R. R., et al (1989). “Abundant alkali sensitive sites in DNA of human and mouse sperm.” Exp Cell Res 184:461-470.
Smith, T. T., Yanagimachi, R. (1989). “Capacitation status of hamster spermatozoa in the oviduct at various times after mating.” J Reprod Fertil 86:255-261.
Szeto, Y. T., Benzie, I. F. F., et al. (2005). “A buccal cell model comet assay: Development and evaluation for human biomonitoring and nutritional studies.” Mutation Research 578(1-2):371-381.
Tas, M., Bacinoglu, S. et al. (2007). “Relationship between bovine fertility and the bumber of spermatozoa penetrating the cervical mucus within straws.” Anim Reprod Sci 101(1-2):18-27.
Tice, R. R, Agurell, E., et al. (2000). “Single cell gel/comet assay: guidelines for in vitro and in vivo genetic toxicology testing.” Environ Mol Mutagen 35:206-221.
Twigg, J., Fulton, N et al. (1998b). “Analysis of the impact of intracellular reactive oxygen species generation on the structural and functional integrity of the human spermatozoa: lipid peroxidation, DNA fragmentation and effectiveness of antioxidants.” Hum Reprod 13:1429-1436.
Vaught, J. B., Henderson, M. K. (2011). “Biological sample collection, processing, storage, and information management.” IARC Sci Publ 3(2)23-42.
Verberckmoes, S., Van Soom, A. (2004). “Storage of fresh bovine semen in a diluent based on the ionic composition of cauda epididymal plasma.” Reprod Dom Anim 39: 410-416.
Viuff, D., Avery, B., et al. (1996). “Transcriptional activity in in vitro produced bovine two- and four-cell embryos.” Mol Reprod Dev 43:171-179.
Ward, S. W. (2013) “Isolation of sperm nuclei and nuclear matrices from the mouse and other rodents.” Methods Mol Biol 927:437-444.
Wilson, R. D., Fricke, P. M., et al. (2005). “In vitro production of bovine embryos using sex-sorted sperm.” Theriogenology doi:10.1016/j.theriogenology.2005.07.007
Xu, J., Chaubal, S. A., Du, F. (2009). “Optimizing IVF with sexed sperm in cattle.” Theriosenolosy 71: 39-47.
Yanagimachi R. (1994). “Mammalian fertilization.” In: The Physiology of Reproduction, 2^{nd} edn (eds E Knobil & JD Neil):189-317. Raven Press, New York.
Yang, X., Jiang, S., Foote, R. H. (1993). “Bovine oocyte development following different oocyte maturation and sperm capacitation procedures.” Mol Repro and Devel 34:94-100.

1. A media formulation comprising a basic salt media and at least one additive,
wherein the basic salt media comprises at least one component selected from the group consisting of sodium chloride (NaCl), potassium chloride (KCl), calcium chloride dihydrate (CaCl₂.2H₂O), magnesium chloride, hexahydrate (MgCl₂.6H₂O), sodium bicarbonate (NaHCO₃), sodium phosphate monobasic dehydrate (NaH₂PO₄2H₂O), potassium dihydrogen phosphate (KH₂PO₄), fructose, sorbitol, bovine serum albumin (BSA), citric acid, and combinations thereof, and
wherein the at least one additive comprises phosphatidylserine (PS) and an NSAID.

2. The media formulation of claim 1, further comprising at least one additive selected from the group consisting of antioxidants, zinc chloride, coenzyme Q10, coumarin compounds, pyranocoumarin compounds, linolenic acid, fatty acids, D-aspartic acid, sodium fluoride, and combinations thereof.

3. The media formulation of claim 1 further comprising a buffer, wherein the buffer is TRIS or HEPES.

4. (canceled)

5. The media formulation of claim 1, wherein the NSAID is acetylsalicylic acid.

6. The media formulation of claim 1, wherein the pyranocoumarin compound is decursin.

7. The media formulation of claim 1, wherein sodium fluoride concentration is about 0 mM to about 6 mM.

8. The media formulation of claim 1, wherein the media formulation enhances activity of a mammalian reproductive cell, enhances zygote/blastocyst formation from germ cells, enhances viability, mobility, and/or fertility of sperm cells, maintains sperm in a fertilization competent state, or alleviates cell loss or DNA damage due to freeze-thaw process.

9. The media formulation of claim 8, wherein fertilization competence is capability of sperm cells exposed to the media formulation for producing pregnancies via artificial insemination, and fertilization, cleavage, or blastocyst conversion both in vitro and in vivo, and/or
the media formulation extends cell viability for at least 24 hours, and/or
said mammalian reproductive cells are selected from the group consisting of gametes, haploid cells, germ cells, sex cells, sperm cells, and egg cells, and/or
the mammalian reproductive cells are derived from ejaculate from a male mammal.

10-12. (canceled)

13. A mammalian reproductive cells composition comprising mammalian reproductive cells and the media formulation of claim 1.

14. The mammalian reproductive cells composition of claim 13 comprising ejaculate from a male mammal, optionally wherein the ejaculate composition is cryopreserved.

15. (canceled)

16. A method of processing mammalian reproductive cells comprising the steps of providing a mammalian reproductive cells sample, processing the mammalian reproductive cells sample, and adding the media formulation of claim 1 to the processed reproductive cells sample to provide a reproductive cells composition.

17. The method of claim 16, wherein the processing comprises at least one step selected from the group consisting of collecting a semen sample, sexing, sorting, separating, freezing, artificial insemination, in vitro fertilization, cooling, transport, and related processes.

18. The method of claim 17, wherein the processing comprises sexing a semen sample to provide a sperm cell composition, and the sexing is accomplished via one or more of droplet sorting, mechanical sorting, micro fluidic processing, microchip processing, jet and air processing, flow cytometry processing, and laser ablation.

19. The method of claim 18, wherein the mammalian reproductive cells are from a bull or boar.

20. (canceled)

21. The method of claim 16, wherein the processed mammalian reproductive cells are gathered in a container, tube, or straw, and/or said mammalian reproductive cells are selected from the group consisting of gametes, haploid cells, germ cells, sex cells, sperm cells, and egg cells.

22. The method of claim 21, wherein said mammalian reproductive cells are sperm cells; and the reproductive cells composition is a sperm cell composition.

23. A method of fertilizing one or more eggs comprising the step of providing an egg obtained from a female mammal, providing the sperm cell composition of claim 22 from a male mammal of the same species as the female mammal, and mixing one or more eggs with the sperm cell composition.

24. The method of claim 23, wherein said mammal is a bull or boar.

25. (canceled)

26. A method of producing an embryo comprising using the sperm cell composition of claim 22 in an assisted reproductive technique, wherein the assisted reproductive technique is selected from the group consisting of in vitro fertilization (IVF), artificial insemination (AI), intracytoplasmic sperm injection (ICSI), multiple ovulation and embryo transfer (MOET), and other embryo transfer techniques.

27-37. (canceled)

38. The media formulation of claim 1, wherein the basic salt media comprises a cauda epididymal plasma (CEP2) media.

39-77. (canceled)
